(19) United States
(12) Patent Application Publication
(10) Pub. No.: US 2003/0158152 A1
(43) Pub. Date: Aug. 21, 2003

(54) Protease inhibitors and their pharmaceutical uses

(76) Inventors: Emerson Poley Pecanha, Rio de Janeiro (BR); Luciana J.O. Figueiredo, Rio de Janeiro (BR); Vera Bongertz, Rio de Janeiro (BR); Octavio Augusto Ceva Antunes, Rio de Janeiro (BR); Amilcar Tanuri, Rio de Janeiro (BR); Rodrigo de Moraes Brindeiro, Rio de Janeiro (BR)

Correspondence Address: Nixon & Vanderhye, 8th Floor, 1100 North Glebe Road, Arlington, VA, 22201-4714, US

(21) Appl. No.: 10/181,817

(22) PCT Filed: Nov. 22, 2001
(86) PCT No.: PCT/BR01/00141

(30) Foreign Application Priority Data
Nov. 23, 2000 (BR) PI0005525-5

(51) Int. Cl. A61K 31/675; A61K 31/44; A61K 31/24; A61K 31/18; A61K 31/165

(52) U.S. Cl. 514/79; 514/357; 514/540; 514/538; 514/601; 514/602; 514/616; 514/114; 546/329; 546/336; 558/190; 564/155; 560/41; 564/84

(57) ABSTRACT

The present invention refers to synthetic protease inhibitors having an axis of symmetry C₂ or pseudo-C₂ characterised by possessing, in the central portion: (1) preferably, a dihydroxyethylene function, which is isosteric with a peptidic bond; (2) a peptidemimetic bridge between the two nitrogens of the main chain and (3) radicals capable of mimetising amino acids. These new protease inhibitors are a base for the preparation of anti-viral formulations capable of inhibiting HIV virus proliferation.

The present invention refers to synthetic protease inhibitors having an axis of symmetry C₂ or pseudo-C₂ characterised by presenting, in the central portion: (1) preferably, a dihydroxyethylene function, which is isosteric with a peptidic bond; (2) a peptidemimetic bridge between the two nitrogens of the main chain and (3) radicals such as benzyl or hydroxybenzyl capable of mimetising amino acids such as, phenylalanine (Phe) or tyrosine (Tyr) groups. These new protease inhibitors are a base for the preparation of anti-viral formulations capable of inhibiting HIV proliferation.

### BACKGROUND OF THE INVENTION

The Acquired Immune Deficiency Syndrome (AIDS) is related to a disease or condition that results in a gradual breakdown of the immunological system, accompanied by a progressive deterioration of the central and peripheral nervous system. Since the beginning of the 80's, when it was recognised, AIDS has been spreading world-wide, having attained epidemical proportions. It is caused by the infection of the human being by a retrovirus, the HIV. The human immunodeficiency virus, or simply HIV, appears to have a special affinity for the human T-4 lymphocyte cell that has a vital role in the immunological system of the body and, in consequence, the immunological system may become inoperative and inefficient against various opportunist diseases, such as pneumocystic pneumonia, Kaposi's sarcoma, cancer of the lymphatic system, amongst others.

The retroviruses causing AIDS contain, as genetic matter, 2 single helix RNAs. After initial infection, a series of essential viral enzymes (reverse transcriptase, RNase-H and integrase) are responsible for the viral RNA transcription into double helix DNA and for the integration of this genetic material into the DNA of the host cell. Thus, once infected by a retrovirus, the host cells and of its progeny acquire the viral genetic information associated to them. Subsequently, the infected cell, using the enzymatic mechanism of the host, is capable of producing new RNA and viral proteins. The retroviral proteins are, then, produced as large polypeptides that need to be modified to produce a new virus. Some of these modifications are undertaken by the enzymes of the host whilst others are executed by enzymes coded by the actual virus. One of the essential retrovirus enzymes is a protease, which is responsible for the transformation of polypeptides into essential enzyme and viral protein structures.

Due to its devastating effect, the HIV protease is one of the most studied retroviral proteases. It is responsible for the selective hydrolysis of the polypeptides, coded by the HIV, “gag” and “gag-pol” to produce the structural proteins that form the viral nucleus as well as essential viral enzymes, including the protease. Mutagenesis studies have demonstrated that mutants with suppression of the protease function do not present infectivity. (see Khol et alli. 1988. Proc. Nat. Acad. 85: 4686; Peng et alli. J. Virol. 63: 2550; Gottlinger et alli. 1989. Proc. Nat. Acad. Sci. 86: 5781; Seelmeier et alli. 1988. Proc. Nat. Acad. Sci. 85: 6612). The structural characterisation of the HIV protease has also been the subject of intense study through X-ray crystallography of recombinant and synthetic proteins (see Navia et alli. 1989. Nature. 337: 615; Lapatto et alli. 1989. Nature 342: 299; Wlodawer et alli. 1989. Science. 245: 616; Miller et alli. 1989. Science. 246: 1149).

The structural characterisation of the HIV protease has revealed that this protein is a C₂ symmetric homodimer which belongs to a class of hydrolytic aspartilprotease enzymes. Most probably, these two characteristics are common to all the retroviral proteases (see (Lapatto et al (1990); Wu et alli. 1990. Arch. Bioch. Biophys. 277: 306).

In the same manner of the other retroviral proteases, the HIV protease cleaves other structural polypeptides at specific sites to release the enzymes and other recently activated structural proteins, rendering, in this manner, the virus capable of replication. It is evident that the inhibition of the HIV protease can avoid the pro-viral integration of the T lymphocytes infected during the initial phases of the HIV life cycle, as well as inhibiting the proteolytic viral processing in the final stages of this cycle. In this manner, the usual treatment for viral diseases normally involves the administration of compounds that inhibit the synthesis of viral DNA.

There have been many works concerning protease inhibitors, specially the symmetric and pseudo-symmetric inhibitors, in view of the confirmation of the potential symmetry of the HIV protease. It is possible to cite, as examples: Moore. 1989. Biochem. Biophys. Res. Commun. 159: 420; Billich. 1988. J. Biol. Chem. 263: 1790S; Richards. 1989. FEBS Lett. 247: 113; Meek et alli. 1990. Nature. 343: 90; McQuade et alli. 1990. Science. 247: 454; Dreyer et alli. 1989. Proc. Nat. Acad. Sci. 86: 9752; Tomaselli et alli. 1990. Biochem. 29: 264; Roberts et alli. 1990. Science. 248: 358; Rich et alli. 1990. J. Med. Chem. 33: 1285; Erickson et alli. 1990. Science. 249: 527; Kempf et alli. 1990. J. Med. Chem. 33: 2687. Apart from these works, various patents have been requested for protease inhibitors, such as: EP 337 714 (Sigal et alli); EP 342 541 e EP 402 646 (Kempf et alli) EP 354 522 (Molling et alli); EP 357 332 (Sigal et alli); EP 346 847 (Handa et alli); EP 356 223 (Desolms et allii); EP 362 002 (Schirlin et alli); EP 352 000 (Dreyer et alli); EP 361 341 (Hanko et alli); EP 374 097 e EP 374 098 (Fassler et alli); WO 90/09191 (Schramm et alli); EP 369 141 (Raddatz et alli); EP 372 537 (Ruger et alli)EP 364 804 (Fung et alli); EP 356 223 (Vacca et alli); EP 361 341 (Hanko et alli); EP 434 365 (Thompson et alli) e EP 492 136 (Babine et alli).

Particularly relevant is the work developed by the group of Kempf et alli (Kempf, D. J., Sowin, T. J., Doherty, E. M., Hannick, S. M., Codavoci, L. M., Henry, R. F., Green, B. E., Spanton, S. G. e Norbeck, D. W. 1992. “Stereocontrolled synthesis of C₂-symmetric and pseudo-C₂-symmetric diamino alcohols and diols for use in HIV protease inhibitors”. J. Org. Chem. 57: 5692-5700). In this work, there is a description of stereochemically controlled synthesis of dibenzyldiamine 1-mono and 2-4 diols with central units of powerful HIV protease C₂-symetric and pseudo-C₂-symetric inhibitors from phenylalanine. Various symmetric and pseudo-symmetric structures, corresponding to the compounds (a) (2S4S)-2,4-diamino-1,5-diphenyl-3-hydroxypentane;

(b) (2S,3R,4R,5S)-2,5-diamino-3,4-dihydroxy-1,6-diphenyl-hexane; (c) (2S,3S,4S,5S)-2,5-diamino-3,4-dihydroxy-1,6-diphenyl-hexane and (d) (2S,3R,4S,5S)-2,5-diamiino-3,4-dihydroxy-1,6-diphenyl-hexane, were studied with the aim of preparing the protease inhibitor (2S,3R,4S,5S)-2,5-di-(N-((N-methyl-N-((2-pyridinyl)methyl)amino)carbonyl)-valinyl-amino)-3,4-dihydroxy-1,6-diphenyl hexane, identified by the code A-77003. This compound showed promise because of its HIV protease inhibiting properties.

Apart from this compound, a large quantity of other potential HIV protease inhibitors were described in the document EP 402 646, including its stereoisomer (2S,3S,4S,5S)-2,5-di-(N-((N-methyl-N-((2-pyridinyl)methyl)amino)carbonyl)-valinyl-amino)-3,4-dihydroxy-1,6-diphenyl hexane. This latter was identified as compound 219 in EP 402 646 and its stereoisomer, named as compound 220, corresponds to the compound A-77003 cited in the article of Kempf et alli (1992). It is worth mentioning that the compounds described in the patent above offer as a possibility for the radical R₃, an alkyl group, but never a hydroxyl group or a protecting group.

Despite the advances attained in the preparation of HIV protease inhibitors, whether in potential or those already in use, there remains a search for new compounds demonstrating more efficiency.

### SUMMERY OF THE INVENTION

The purpose of the present invention is to provide new and efficient C₂-symetric HIV protease inhibitors having the general formula I.

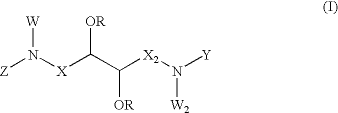

where:

Z and Y are independently selected from CHR₂R₃; CHR₄COOR₅; CHR₄CONHR₆ and CHR₄C(O)NHN═CR₇R₈

R₆ is selected from (NH₂), CHR₄COOR₅, hydrogen, aryl, substituted aryl, arylalkyl, substituted arylalkyl, heterocycles, alkyl heterocycles and lower alkyl

R₂, R₃, R₄, R₇, R₈ are independently selected from hydrogen, aryl, substituted aryl, arylalkyl, substituted arylalkyl, heterocycles, alkyl heterocycles and lower alkyl

R₅ is an lower alkyl or hydrogen

W and W₂ are independently selected from hydrogen, lower alkyl, carbonylalkyl, carbonylaryl, alkylsulphone, arylsulphone, substituted arylsulphone

R is hydrogen or a protecting group and

X and X₂ are independently selected from CH₂ and CO.

The term lower alkyl means alkyl radicals with straight or branched chains containing from 1 to 6 atoms of carbon, including, but not limited to methyl, ethyl, n-propyl, iso-propyl, n-butyl and iso-butyl, sec-butyl, n-pentyl.

The term protecting group refers to groups which protect the hydroxyl groups against undesirable reactions during the synthesis stages or to avoid the attack by exopeptidases of the final compounds or with the aim of increasing the solubility of the final compounds including, but not limited to, acyl, acetyl, phosphoryl pivaloyl, t-butylacetyl, benzoyl, substituted methyl ethers, such as methoxymethyl, benzyloxymethyl, 2-methoxy-ethoxy-methyl, substituted ethyl ethers, such as 2,2,2-trichlorolethyl, and esters prepared through the reaction of hydroxyl group with carboxylic acid, for example, acetate, propionate, benzoate, amongst others.

The term aryl, as employed here, consists of carbocyclic bicyclic or monocyclic ring systems possessing one or more aromatic rings, including, but not limited to, phenyl, naphthyl, and tetrahydronaphthyl, amongst others. The aryl groups may be unsubstituted or substituted by one, two or three substituents, independently selected, but not limited to, a lower alkyl, haloalkyl, hydroxy, nitro, amine, carboxy, mercaptan.

The term arylalkyl refers to an aryl group linked to lower alkyl radical, including, but not limited to, benzyl, p-hydroxybenzyl, α-naphthylmethyl, amongst others.

The term alkylsulphone refers to a sulphone group linked to lower alkyl radical, including, but not limited to, methylsulphone, n-propylsulphone, isopropylsulphone, n-butylsulphone, isobutylsulphone.

The term arylsulphone refers to a sulphone group linked to an aryl radical, including, but not limited to, benzenesulphone, 4-methyl-benzenesulphone, 4-amino-benzenesulphone, 4-hydroxy-benzene sulphone.

The term carbonaryl refers to a carbonyl group linked to an aryl radical, including, but not limited to, benzenecarbonyl, 4-methyl-benzenecarbonyl, 4-amino-benzenecarbonyl, 4-hydroxy-benzenecarbonyl.

The term carbonalkyl refers to a carbonyl group linked to lower alkyl radical, including, but not limited to, acetyl, propionyl, n-butyril, isobutyril, n-valeroyl, isovaleroyl.

The term heterocyclic ring or heterocycle refers to any ring of 3 or 4 members containing a heteroatom selected from oxygen, nitrogen and sulphur; or to 5- or 6-membered ring containing one, two or three atoms of nitrogen; an atom of nitrogen and an atom of sulphur; or an atom of nitrogen and an atom of oxygen. The 5-membered ring possesses from 0 to 2 double bonds and the 6-membered ring possesses from 0 to 3 double bonds. The heteroatoms of nitrogen and sulphur may be, optionally, oxidized. The term heterocycle also includes bicyclic groups in which any of the above heterocyclic rings is conjugated to a benzene or a cyclohexane or any other heterocyclic ring. Heterocyclic rings include, but are not limited to, pyrrolyl, pyrrolinyl, imidazolyl, imidazolinyl, pyridyl, piperidinyl, oxazolyl, thiazolyl, quinolyl, isoquinolyl, indolyl and furyl.

The heterocycles may be unsubstituted, mono or di-substituted with substituents independently selected from hydroxy, halo, oxo, amino, alkylamino, cycloalkyl, carboxyl and lower alkyl.

The term alkyl heterocycle employed here refers to heterocyclic groups linked to lower alkyl radicals including, but not limited to, imidazolylmethyl, thiazolylmethyl, pyridylmethyl.

The chiral centres of the compounds of the invention may be racemic or asymmetrical. Racemic mixtures, mixtures of diasteroisomers, as well as singular diasteroisomers of the compounds of the invention are included within the scope of the present invention. The definition of the “R” and “S” configurations are contained in the recommendations of the IUPAC of 1974 (Fundamental Stereochemistry, Pure Appl. Chem. 45.13-30. 1976).

The terms “Ala”, “Ile”, “Leu”, “Phe”, “Val”, “Trp” and “Tyr”, as employed here, refer to alanine, isoleucine, leucine, phenylalanine, valine, tryptophan and tyrosine, respectively. Generally, the abbreviation of the amino acids used here follow the IUPAC nomenclature.

A first embodiment of the present invention concerns compounds with HIV protease inhibiting properties, having a peptidemimetic chain between the two atoms of nitrogen of the main chain and preferentially a central dihydroxyethylenic function as defined by the general formula (I).

In a second embodiment, anti-HIV formulations based on the protease inhibiting compounds of the invention are provided.

In a third embodiment, anti-HIV formulations based on the protease inhibiting compounds of the invention, in association with other compounds that inhibit the HIV protease, are provided.

### DETAILED DESCRIPTION OF THE INVENTION

As mentioned above, the confirmation of the existence of an axis of symmetry C₂ in the HIV protease led to a study of the behaviour of this enzyme, as well as to research and synthesis of potential inhibitors, including the structural requirements necessary for a sufficient bioavailability of drugs based on these inhibitors. (see Abdel-Meguid, S. S. et alli. 1994. “An orally bioavailable HIV-1 protease inhibitor containing an imidazole-derived peptide bond replacement: crystallographic and pharmacokinetic analysis”. Biochemistry. 33: 11671-11677).

The determination of the manner in which the HIV protease acts to enable a hydrolysis of peptides is another important characteristic of the research for efficient anti-HIV drugs (see Fitzgerald, P. M. D. et alli. 1990. “Crystallographic analysis of a complex between Human Immunodeficiency virus type 1 protease and acety-pepstatin at 2.0 Å Resolution”. J. Biol. Chem. 265: 14209-14219; Medzihradszky, K. et alli. 1970. “Effect of secondary enzime-substrate interactions on the cleavage of synthetic peptides by pepsin”. Biochemistry. 9: 1154-1162). In this direction, Fitzgerald et alli (1990) proposed the diagrams of the hydrogen bridges and the non-linked interactions showing the involvement of the amino acid (S)-aspartic acid, which represents an active HIV protease site. The interaction enzyme-substrate may be established through the potential hydrogen bridges, the most part of them with distances ranging from 240 to 310 pm. In the case of the interaction between Asp25 (O₁ and O₂) and Sta4-OH, the distances are slightly larger (330 and 390 pm).

The characterization of the HIV protease inhibitor structures of the present invention was undertaken both from empirical analogy with known HIV protease inhibitors and also from knowledge of the possible interaction with the active site of this enzyme. Initially, theoretic calculations of quantum mechanics at a semi-empirical level (programme AM1 (Austin Model 1) under the software MOPAC7 (Molecular Orbital Package 7)) were employed to determine the geometries of the potential HIV protease inhibitors of the present invention.

The chemical variables selected to relate the chemical structure with the biological activity were: enthalphy, molecular radius, charge on the oxygen of the carbonyl, lengths of the N—H and O—H bonds, dipolar moment, energy of the molecular orbital of HOMO and partition coefficient of water/octanol. The choice of the variables was made based on the crystallographical data of the HIV-1 co-crystallised with inhibitors of the hydroxyethylene type and intend to describe the interactions involved at the site.

The central hydroxyethylene portion and the amino groups present in the inhibitor interact with the catalytic amino acids Asp25 and Asp25′ of the enzyme (HIV protease) whilst the carbonyl groups of the inhibitor are receptive to additional hydrogen bonds. The variables: lengths of the amino (N—H) and hydroxy-central (O—H) bonds, dipolar moment (μ), charge on the oxygen of the carbonyl (Q_{co}), energy of the boundary orbital of HOMO (E_{HOMO}) and the molecular diameter were employed to describe general aspects at the site of interaction. (O—H), (N—H) and (Q_{co}) are directly related to the hydrogen bonds between the inhibitor and the enzyme. The partition coefficient of water/octanol (logP) is a typical variable of structure/activity studies, related to the hydrophobic/hydrophilic profile of the inhibitor. The energy of the boundary orbital (E_{HOMO}) is a classificatory variable that describes the potential of these inhibitors to act as nucleophiles (as should be expected by the interactions at the active site of the enzyme). The other classificatory variable is enthalpy (ΔH) which classifies the inhibitors in terms of their thermodynamic stability.

All data were obtained by theoretical calculations, with the partition coefficient of water/octanol being obtained using the ACD/LogP Software program (ACD/Labs® for MS Windows® at http://www.acdlabs.com) and the remaining data being calculated with the AM1 program. The physicochemical parameters obtained by theoretical calculations were analysed in multivariate manner with the methodologies: Principal Component Analysis (PCA), Hierarchic Cluster Analysis (HCA), as well as the classificatory analyses SIMCA (Soft Independent Modelling of Class Analogy) and KNN (K-Nearest Neighbour). The analysis was done comparatively amongst the HIV protease inhibitors of the invention and already known inhibitors. SIMCA and KNN are methods capable of classifying known and unknown samples in categories based on similarities encountered in the set of variables. SIMCA is a classificatory method based on similarities of the principal components whilst KNN uses multivariated space for a classification in groups of similar objects by their localisation. These two methodologies generate similar and very often complementary results.

These methodologies allow relating the biological activity to the chemical structure, which saves wasting time and material with the synthesis of compounds which, in many cases, do not conform to the requirements of the biological activity. Thus, the multivariate chemometric analysis simplifies this complex situation of variables and the desired information can be obtained simultaneously by observing the tendency of the inhibitors to be separated into groups and the variables of greater importance in this separation.

Mathematically, the best manner of representing a set of data with multivariated origin is to build a matrix that relates variables and samples (or objects). In the case of the inhibitors studied, this matrix is composed of 45 inhibitors and of eight physicochemical variables selected for the training set (known inhibitors) and 18 compounds selected from the possibilities included in the general formula I, as well as the eight variables selected for the training set.

Each object is then placed in an n-dimensional space (where n is the number of variables). The PCA method permits the projection of a space of superior order in two or three dimensions with a minimum loss of statistical information. The axes of the co-ordinates of the space of the original n order are rotated until they reach the maximum direction of variance, thus, therefore, obtaining the axis of the first principal component. The principal components that follow are constructed orthogonally to the former one and in the direction of the maximum residue of variance remaining.

The two first principal components were used and represent 55% of the total variance of the data. It is possible to note the separation into three principal groups, where one of them corresponds to structures of the invention, selected from the possibilities foreseen in the general formula I. In this case, 18 possible structures were analysed. The most important parameters for this separation were: molecular volume, charge on the carbon of the carbonyl, dipolar moment, energy of the HOMO, length of the N—H bond and coefficient of the water/octanol ratio.

The last chemometric analysis undertaken was the classification of the test set (protease inhibitors of the invention) by the SIMCA and KNN methods. Once again, auto evaluated data having three principal components were used. Category 1 includes 12 inhibitors with Ki>10 nM, category 2 includes the 12 remaining inhibitors with Ki<10 nM and category 3 is composed of the 18 potential inhibitors.

The percentage of accuracy for the classification by the SIMCA method was of 89% for the three categories. Another interesting result shows that the group of inhibitors of category 3 is closer to the good inhibitors (category 2 with Ki<10 nM) than to the bad inhibitors (category 1 with Ki>10 nM). The classification by KNN showed an accuracy of 79%.

The studies of the molecular modelling by docking simulating the interaction between the inhibitors of the present invention and the HIV-1 protease enzyme were done by using the DOCK, version 4.0 program.

The crystallographic structures of the HIV-1 protease, whether isolated or co-crystallized, were obtained from the data base known to those versed in the technique, in this case the Protein Data Bank (PDB). In the case of the inhibitors, the structures were obtained by AM1 calculations.

This theoretical methodology permitted designing the protease inhibitors of the invention that comply with the general formula I.

From the data obtained above it was then possible to pass on to the synthesis stage of the HIV protease inhibitors foreseen in the general formula I.

Table 1 below presents some preferred compounds of the invention with formula A—B—C, where A is defined as (X)N(W) (Z), B is defined as (CHOR)₂ and C is defined as (X₂)N(W₂)(Y).

TABLE 1
Preferred Compounds

| Composto | A | B | C |
|---|---|---|---|
| 1a | 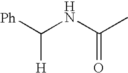 | 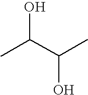 | 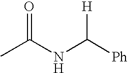 |
| 1b (S,S,S,S) | 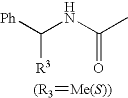 | 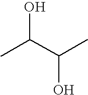 | 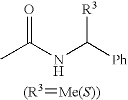 |
| 1c (R,S,S,R) | 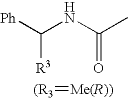 | 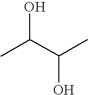 | 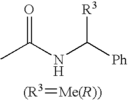 |
| 3a | 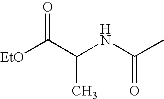 | 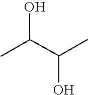 | 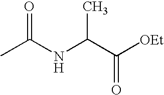 |
| 3b | 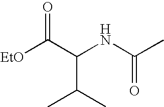 | 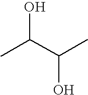 | 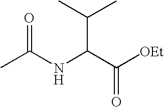 |
| 3c | 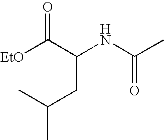 | 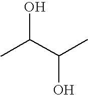 | 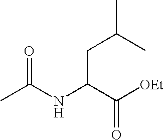 |
| 3d | 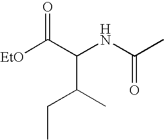 | 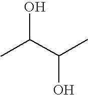 | 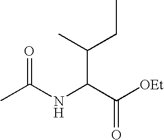 |
| 3e | 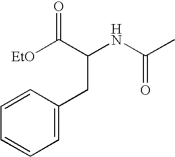 | 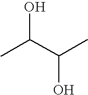 | 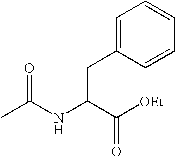 |
| 3f | 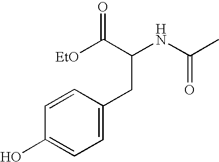 | 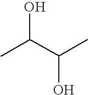 | 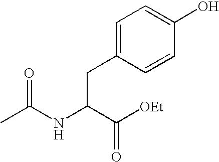 |
| 3g | 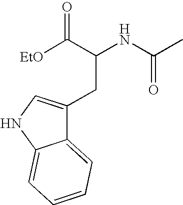 | 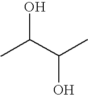 | 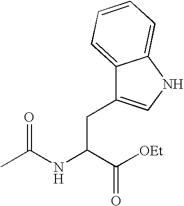 |
| 2a | 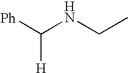 | 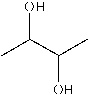 | 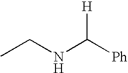 |
| 2b (S,S,S,S) | 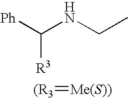 | 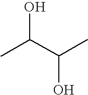 | 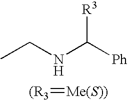 |
| 2c (R,S,S,R) | 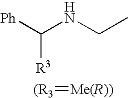 | 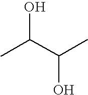 | 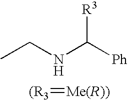 |
| 4a | 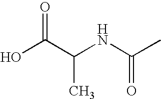 | 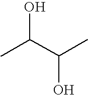 | 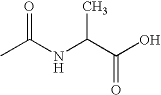 |
| 4b | 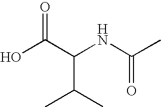 | 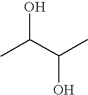 | 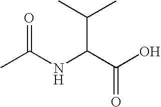 |
| 4c | 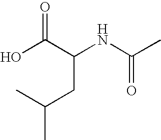 | 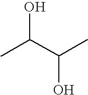 | 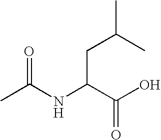 |
| 4d | 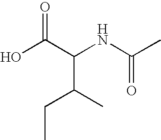 | 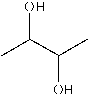 | 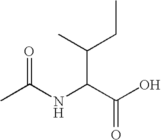 |
| 4e | 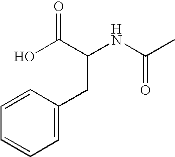 | 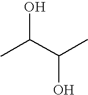 | 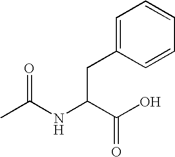 |
| 4f | 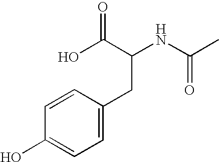 | 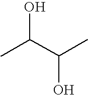 | 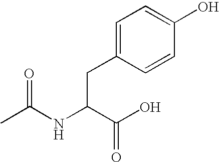 |
| 4g | 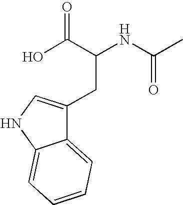 | 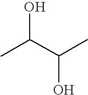 | 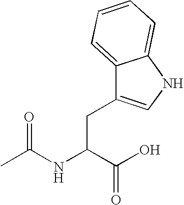 |
| 5a | 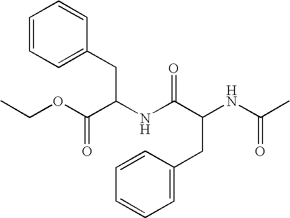 | 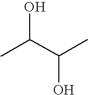 | 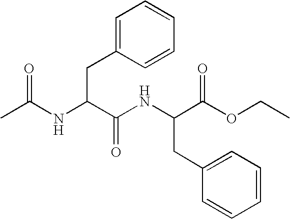 |
| 5b | 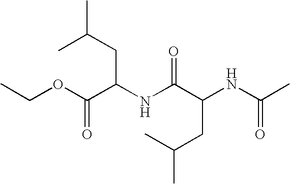 | 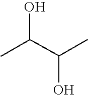 | 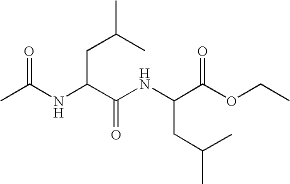 |
| 5c | 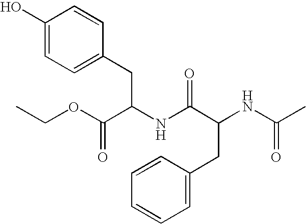 | 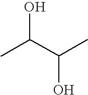 | 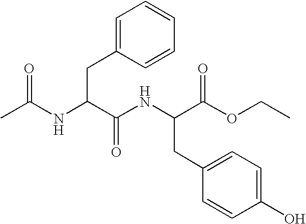 |
| 5d | 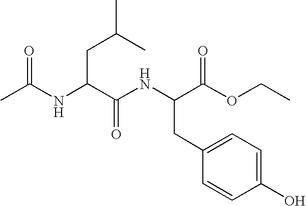 | 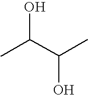 | 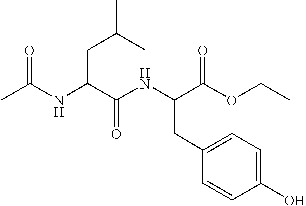 |
| 8a | 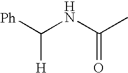 | 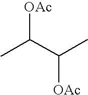 | 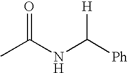 |
| 8b (S,S,S,S) | 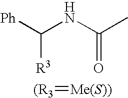 | 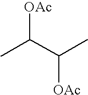 | 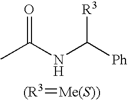 |
| 8c (R,S,S,R) | 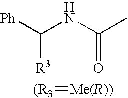 | 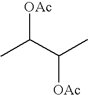 | 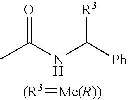 |
| 10a | 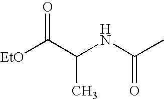 | 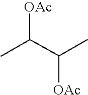 | 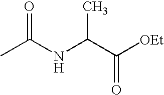 |
| 10b | 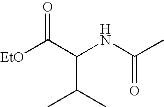 | 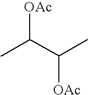 | 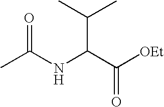 |
| 10c | 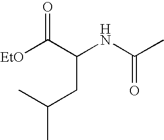 | 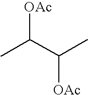 | 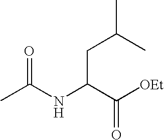 |
| 10d | 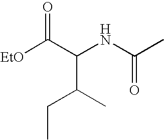 | 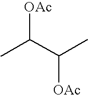 | 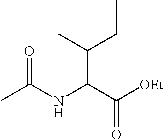 |
| 10e | 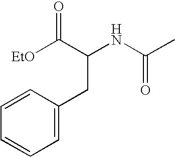 | 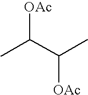 | 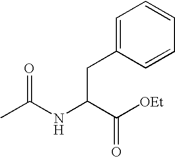 |
| 10f | 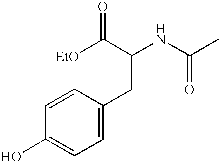 | 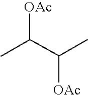 | 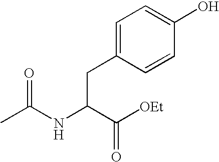 |
| 10g | 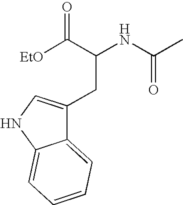 | 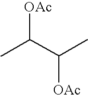 | 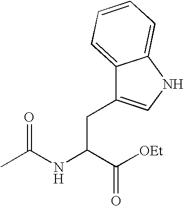 |
| 11a | 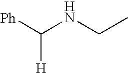 | 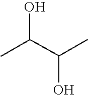 | 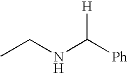 |
| 11b (S,S,S,S) | 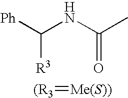 | 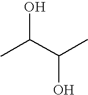 | 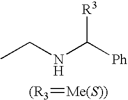 |
| 11c (R,S,S,R) | 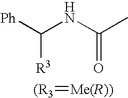 | 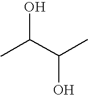 | 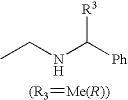 |
| 23 | 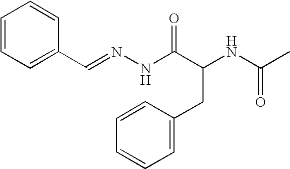 | 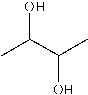 | 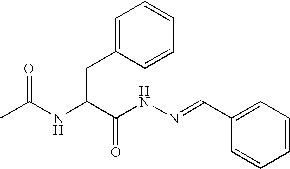 |
| 24 | 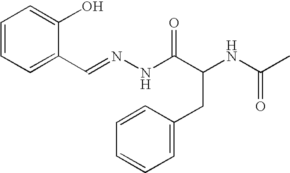 | 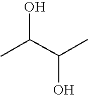 | 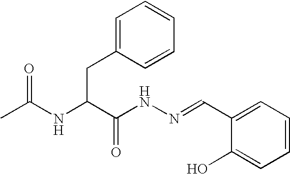 |

The compounds of the present invention present adequate structural characteristics for a bonding to a target-enzyme, i.e. the presence of non hydrolysable group, isostere to the peptidic bond, represented by the dihydroxy-ethylene group, capable of interacting through hydrogen bonds with the catalytic site of the enzyme; groups capable of interacting through hydrophobic bonds with the recognition sites S₁ and S₁′ in the compounds (1a-1c), (2a-2c), (3a-3g), (4a-4g) and with sites of S₁, S₁′, S₂ and S₂′ in the derivatives (5a-5d), all presented in Table 1.

Considering, also, the nature of homodimer with an axis of symmetry C₂ presented by the target macromolecule, the derivatives (1a-1c), (2a-2c), (3a-3g), (4a-4g) and (5a-5d) that present a c₂ axis, presented a good structural complementary with the enzyme, and consequently a good constant of affinity as well as an adequate pharmacological potency. The ethyl esters (3a-3g) present a partition coefficient of lipids/water more adequate to cellular membrane penetration than the acids (4a-4g), thus representing major synthetic targets.

The first stage for obtaining the derivatives of the invention consists of protecting the hydroxyl groups of tartaric acid. The protection of the hydroxyl groups against undesirable reactions during the synthesis stages or to avoid the attack by exopeptidases of the final compounds or with the aim of increasing the solubility of the final compounds involving the reaction with, but not limited to, acyl, acetyl, phosphoryl pivaloyl, t-butylacetyl, benzoyl, substituted methyl ethers, such as methoxymethyl, benzyloxymethyl, 2-methoxy-ethoxy-methyl, substituted ethyl ethers, such as 2,2,2-trichloroethyl, and esters prepared through the reaction of the hydroxyl group with a carboxylic acid group, for example, acetate, propionate, benzoate, amongst others. The acetylation of tartaric acid was one of the protection strategies employed, since the hydroxyl groups could easily be released through hydrolysis in mild conditions, in which the amide bonds present in the peptidemimetic derivatives would be inert (Paquette et al, 1999). The derivative (6) is obtained from D-tartaric acid (7) at 85% yield, through treatment with acetyl chloride, under reflux, during 48 h, followed by recrystallisation (Almeida et al, 1992).

After the protection of the derivative of tartaric acid (6), the following stage is the formation of the acid chloride (I), and its coupling in situ with the amines necessary for obtaining the derivatives of interest. The formation of the acid chloride (I) was obtained by the treatment of the compound (6) with 1.5 to 2.0 eq. of oxalyl chloride, for around 2 h at room temperature, in a nonpolar organic solvent selected from the group comprising chloroform, dichloromethane, dichloroethane, diethyl ether, toluene, amongst other nonpolar organic solvents known to those versed in the subject, in the presence of catalytic quantities of N,N-dimethylformamide. The intermediate (|) was coupled in situ with the necessary amines (1.2 eq.) in a non polar organic solvent selected amongst those mentioned above, at room temperature for around 30 min in the presence of 1.5 to 2.0 eq. of triethylamine. The results obtained, for some selected examples are shown in Table 2.

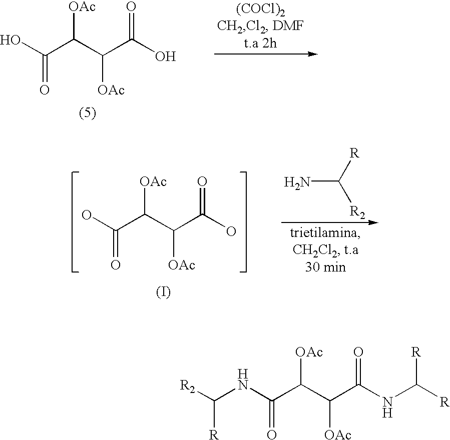

TABLE 2
Preparation of compounds (8a-8c) and (10a-10g)

| | | | YIELD | |
|---|---|---|---|---|
| COMPOUND | R | R₂ | (%) | PF (° C.) |
| 8a | H | PHENYL | 96 | 184-185 |
| 8b | CH₃ (S) | PHENYL | 90 | 226-227 |
| 8b | CH₃ (S) | PHENYL | 90 | 212-213 |
| 10a | CH₃ | CARBETOXY | 85 | 170-171 |
| 10b | 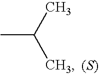 | CARBETOXY | 93 | 147-148 |
| 10c | 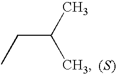 | CARBETOXY | 94 | 137-138 |
| 10d | 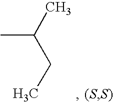 | CARBETOXY | 93 | 156-157 |
| 10e | 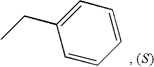 | CARBETOXY | 95 | 159-160 |
| 10f | 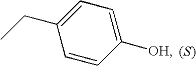 | CARBETOXY | 94 | 210-211 |
| 10g | 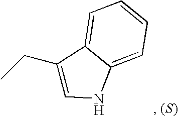 | CARBETOXY | 78 | 103-105 |

The liberation of the hydroxyl groups in the tartaric acid derivatives (8a-8g) and (10a-10g) was achieved through the treatment with an alcohol, for example, through an ethanolysis of the acetyl groups, employing a strong acid, such as catalytic amount of sulphuric acid in absolute ethanol, under reflux during ca. 4 h. The diols (1a-1c) and (3a-3g) were obtained in yields between 55 and 83% (Table 3).

TABLE 3
Acid ethanolysis of (8a-8c) and (10a-10g)

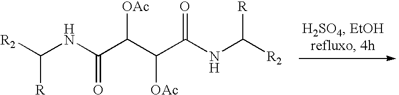

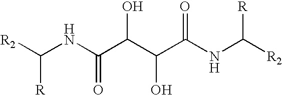

| | | | YIELD | |
|---|---|---|---|---|
| COMPOUND | R | R₂ | (%) | PF (° C.) |
| 1a | H | PHENYL | 83 | 198-200 |
| 1b | CH₃ (S) | PHENYL | 82 | 130-131 |
| 1c | CH₃ (R) | PHENYL | 83 | 144-146 |
| 3a | CH₃ | CARBETOXY | 60 | 102-103 |
| 3b | 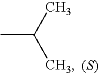 | CARBETOXY | 82 | Oil |
| 3c | 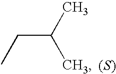 | CARBETOXY | 81 | Oil |
| 3d | 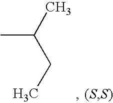 | CARBETOXY | 81 | Oil |
| 3e | 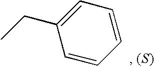 | CARBETOXY | 80 | 138-140 |
| 3f | 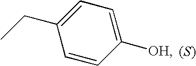 | CARBETOXY | 77 | 104-105 |
| 3g | 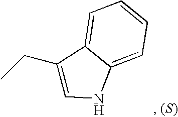 | CARBETOXY | 55 | 114-116 |

The aminoalcohols (2a-2c) were obtained through the reduction of the protected diamides (8a-8c), employing LiAlH₄ (3eq.) under reflux of THF, during ca. 48 h. These conditions provide the target compounds (2a-2c) in yields between 57 and 62%, after separation by column chromatography with silica gel. Additionally, the mono amides (11a-11c) were isolated at a 10-12% yield (Table 4). The formation of the derivatives (11a-11c), unexpected under these vigorous reaction conditions, cannot be avoided even by the extension of the reaction time to 72 h. However, compounds (11a-11c) present the minimum structural requirements for an adequate interaction with the HIV-PR.

TABLE 4
Reduction of the compounds (8a-8c) with LiAlH₄.

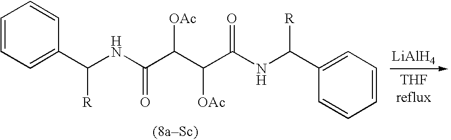

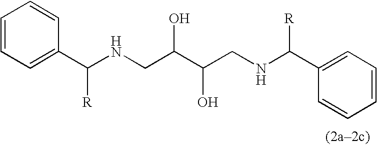

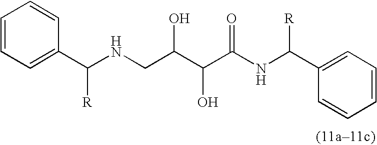

| | Time | | | | |
|---|---|---|---|---|---|
| R | (h) | Compound | Yield | Compound | Yield |
| H | 48 | 2a | 57% | 11a | 10% |
| H | 72 | 2a | 59% | 11a | 10% |
| CH₃ (R) | 48 | 2c | 62% | 11c | 12% |
| CH₃ (R) | 72 | 2c | 63% | 11c | 12% |
| CH₃ (S) | 48 | 2b | 62% | 11b | 12% |
| CH₃ (S) | 72 | 2b | 64% | 11b | 11% |

The compounds of the present invention may be used in the inhibition of the HIV protease, in the prevention or treatment of infection caused by HIV, as well as in the treatment of the subsequent pathological conditions characteristic of AIDS. The terms “prevention” and “treatment” include but are not limited to the treatment of a wide range of infectious conditions due to HIV, symptomatic and asymptomatic, such as AIDS, ARC (AIDS Related Complex), whether real or potential occurring from exposure to HIV.

The total daily dose administered, whether single or divided, may vary, for example, between 0.1 and 100 mg/kg of body weight, per day.

The quantity of the active ingredient to be combined with an acceptable pharmaceutical vehicle, so as to produce the form of single dose, will depend on the organism being treated and the chosen method of administration. The active ingredient, preferentially, will comprise from 0.1 to 99% in weight of the formulation. However, preferentially, it should be present at a concentration varying between 0.25 and 99% in weight of the formulation.

However, it must be understood that the specific level of the dose for any patient will depend on a variety of factors, including the activity of the specific compound used, age, body weight, overall clinical condition, sex, diet, time and means of administration, rate of excretion, association with other drugs and severity of the disease to be treated.

In the present invention, the compounds with symmetry C₂ may occur as racemic mixtures or as isolated stereoisomers, with the latter being preferred.

The compounds of the present invention may be used in the form of salts derived from organic or inorganic acids. These salts include, but are not limited to, acetate, adipate, alginate, citrate, benzoate, aspartate, bisulphate, dodecylsulphate, butyrate, ethylsulphate, glycerophosphate, mesylate, propionate, lactate, amongst others.

Examples of acids that may be employed to form pharmaceutically acceptable salts include, but are not limited to, inorganic acids such as sulphuric acid, hydrochloric acid and phosphoric acid, and, as examples of organic acids, oxalic acid, maleic acid, citric acid, methylsulphonic acid and succinic acid. Other salts include those with alkaline metals or alkaline earth metals, such as sodium, potassium, calcium or magnesium or also with organic basis.

The compounds of the present invention may also be used under the form of esters. Such esters function as pro drugs of the respective compounds of the present invention and serve to increase the solubility of these compounds in the gastrointestinal tract. These esters also serve to increase the solubility of the respective compounds of the present invention when administered intravenously. These compounds are metabolised in vivo to provide the substituted hydroxyl compound of the general formula I. These pro-drugs are prepared by the reaction of substituted hydroxyl compounds of formula I with, for example, an activated aminoacyl group or phosphoryl group, amongst others. The resulting product is, then, released to provide the desired pro drug. Furthermore, it must be stressed that the compound of the general formula I possessing the protected hydroxyl may also be employed as a pro drug.

The protease inhibitors of the invention are used as a single active ingredient, or in association with other inhibitors, in formulations containing pharmaceutically acceptable non-toxic vehicles and adjuvants, which are prepared in accordance with known and standardised techniques.

In the case of oral administration, the non-active components include excipients, bonding agents, desintegrators, diluents, lubricants, controlled release agents, etc., such as microcrystalline cellulose, alginic acid or sodium alginate, methylcellulose, dicalcium phosphate, starches, magnesium stearate.

In the injectable form, acceptable diluents and parenteral solvents may be used, as well as other non-toxic components, such as suspension agents, oils, synthetic mono- and diglycerides, fatty acids etc.

The present invention is described in detail through the examples presented below, it is necessary to point out that the invention is not limited to these examples, but also includes variations and modifications within the limits in which it functions.

### EXAMPLE 1

### Preparation of the Acid 2,3-diacetoxy -(2R,3R)-butanedioic (Compound 17)

A solution of L-tartaric acid (20.00 g, 133.33 mmols) in acetyl chloride (200 ml) was kept under magnetic stirring at reflux temperature for 48 h. After this period, the reaction mixture was evaporated and the solid residue obtained was recrystallized in AcOEt/Hexane, providing the compound (17) (26.52 g, 113.33 mmols) at 85% yield as a white hygroscopic crystalline solid: PF 109-110° C. [α]_{D}=+95.0 (c=1.00 H₂O), ¹H RMN (CDCl₃, 200 MHz) δ5.72 (s, 1H), 2.21 (s,3H); ¹³C RMN (CDCl₃, 50 MHz) δ169.8, 163.4, 72.2, 20.2; IR (cm⁻¹) 3300, 2942, 1743, 1693, 1239, 1089.

### EXAMPLE 2

### Preparation of the Acid 2,3-diacetoxy-2S,3S)-butanedioic (Compound 6)

The compound (6) (26.52 g, 113.33 mmols) was obtained from D-tartaric acid (20.00 g, 133.33 mmols) through the same procedure described for obtaining (17) at 85% yield as a white hygroscopic crystalline solid: PF 109-110° C. [α]_{D}=−95.2 (c=1.00 H₂O), ¹H RMN (CDCl₃, 200 MHz) δ5.72 (s, 1H), 2.21 (s,3H); ¹³C RMN (CDCl₃, 50 MHz) δ169.8, 163.4, 72.2, 20.2; IR (cm⁻¹) 3300, 2942, 1741, 1703, 1239, 1089.

### EXAMPLE 3

### Preparation of 1N,4N-dibenzyl-2,3-diacetoxy-(2R,3R)-butanediamide (Compound 20)

Oxalyl chloride (1.62 g, 12.8 mmols) was added for a period of 10 minutes to a solution of compound (17) (1.00 g, 4.27 mmols), and DMF (0.2 ml) of anhydrous dichloromethane at 0° C. under magnetic stirring in an argon atmosphere. After a period of 2 h at room temperature, the solution was evaporated under vacuum, and the yellowish solid residue was recovered in dichloromethane (20 ml), and added for a period of 20 minutes to a mixture of benzylamine (1.10 g, 10.3 mmols) and triethylamine (1.29 g, 12.8 mmols), at room temperature. At 30 minutes of magnetic strring, the mixture was concentrated under vacuum, recovered in AcoEt (100 ml) and extracted with aqueous HCl (2×70 ml). The organic phase was washed with a saturated solution of sodium chloride (50 ml), dried with sodium sulphate, and evaporated under vacuum, providing the diamide (20) (1.69 g, 4.10 mmols) at 96% yield, as a white solid. Analytically pure samples may be obtained by recrystallization in AcoEt/hexane: PF 184-185° C. [α]_{D}=+5.0 (c=1.20, CH₃OH). ¹H RMN (CDCl₃, 200 MHz) δ7.26 (m, 5H), 6.43 (m, 1H), 5.69 (s, 1H), 4.52 (dd, J=6.5, 14.8 Hz, 1H), 4.29 (dd, J=5.1, 14.8 Hz, 1H), 2.05 (s, 3H); ¹³C RMN (CDCl₃, 50 MHz) δ169.1, 166.1, 137.6, 128.8, 127.7, 72.5, 43.5, 20.4; IR (cm⁻¹) 3321, 3088, 3033, 2942, 1474, 1683, 1660, 1533, 1239, 1089, 749, 702.

### EXAMPLE 4

### Preparation of 1N,4N-dibenzyl-2,3-diacetoxy-(2S,3S)-butanediamide (Compound 8a)

Compound (8a) (1.67 g, 4.06 mols) was obtained from 2,3-diacetoxy-(2S,3S)-butanedioic (6) (1.00 g, 4.27 mmols) and benzylamine (10 3 mmols), by the same procedure described for obtaining (20), with 95% yield, as a white solid. Analytically pure samples may be obtained by recrystallization in AcoEt/hexane: PF 184-185° C. [α]_{D}=−4.8 (c=1.12, CH₃OH). ¹H RMN (CDCl₃, 200 MHz) δ7.19 (m, 5H), 6.62 (m, 1H), 5.64 (s, 1H), 4.41 (dd, J=6.5, 14.8 Hz, 1H), 4.17 (dd, J=5.1, 14.8 Hz, 1H), 1.97 (s, 3H); ¹³C RMN (CDCl₃, 50 MHz) δ169.4, 166.3, 137.7, 128.9, 127.9, 72.6, 43.6, 20.7; IR (cm⁻¹) 3321, 3089, 3032, 2983, 1747, 1683, 1659, 1532, 1239, 749, 702.

### EXAMPLE 5

### Preparation of 1N,4N-di[1-phenyl-(1S)-ethyl]-2,3-diacetoxy-(2S,3S)-butanediamide (Compound 8b)

Compound (8b) (1.69 g, 3.84 mmols) was obtained from 2,3-diacetoxy-(2S,3S)-butanedioic acid (6) (1.00 g, 4.27 mmols) and (S)-α-methylbenzylamine (1.25 g, 10.3 mmols), by the same procedure described for obtaining (20), with 90% yield, as a white solid. Analytically pure samples may be obtained by recrystallization in acetone-water: PF 226-227° C. [α]_{D}=−53.7 (c=1.08, CH₂Cl₂). ¹H RMN (CDCl₃, 200 MHz) δ7.25 (m, 5H), 7.08 (br s, 1H), 5.64 (s, 1H), 4.99 (m, 1H), 1.94 (s, 3H), 1.42 (d, J=6.7 Hz, 3H); ¹³C RMN (CDCl₃, 50 MHz) δ168.2, 164.1, 141.4, 127.2, 126.0, 124.8, 71.3, 47.8, 20.0, 19.0; IR (cm⁻¹) 3255, 3063, 3029, 2978, 1755, 1649, 1544, 1208, 1055, 756, 698.

### EXAMPLE 6

### Preparation of 1N,4N-di[1-phenyl-(1R)-ethyl]-2,3-diacetoxy-(2S,3S)-butanediamide (Compound 8c)

Compound (8c) (1.69 g, 3.84 mmols) was obtained from 2,3-diacetoxy-(2S,3S)-butanedioic acid(6) (1.00 g, 4.27 mmols) and (R)-α-methylbenzylamine (1.25 g, 10.3 mmols), by the same procedure described for obtaining (20), with 90% yield, as a white solid. Analytically pure samples may be obtained by recrystallization in AcOEt/hexane: PF 212-213° C. [α]_{D}=+41.8 (c=0.98, CH₂Cl₂). ¹H RMN (CDCl₃, 200 MHz) δ7.27 (m, 5H), 6.77 (d, J=8.0 Hz, 1H), 5.70 (s, 1H), 5.05 (m, 1H), 1.94 (s, 3H), 1.44 (d, J=7.0 Hz, 3H); ¹³C RMN (CDCl₃, 50 MHz) δ169.4, 165.5, 142.5, 128.9, 127.7, 126.4, 72.7, 48.9, 21.6, 20.6; IR (cm⁻¹) 3359, 3087, 3031, 2986, 2942, 1756, 1660, 1529, 1208, 1057, 765, 701.

### EXAMPLE 7

### Preparation of 1N,4N-di[1-carbetoxy-3-methyl-(1S)-butyl]-2,3-diacetoxy-(2S,3S)-butanediamide (Compound 10c).

Compound (10c) (2.05 g, 3.97 mmols) was obtained from 2,3-diacetoxy-(2S,3S)-butanedioic acid(6) (1,00 g, 4,27 mmols) and ethyl ester of leucine (1,64 g, 10,3 mmols), by the same procedure described for obtaining (20), with 93% yield, as a white solid. Analytically pure samples may be obtained by recrystallization in AcOEt/hexane: PF 156-157° C. [α]_{D}=−14.0 (c=1.00, CH₂Cl₂). ¹H RMN (CDCl₃, 200 MHz) δ6.64 (d, J=8.4 Hz, 1H), 5.65 (s, 1H), 4.59 (m, 1H), 4.19 (q, J=7.1 Hz, 2H), 2.19 (s, 3H), 1.62 (m, 3H), 1.28 (t, J=7.1 Hz, 3H), 0.95 (d, J=5.4 Hz, 6H); ¹³C RMN (CDCl₃, 50 MHz) δ172.7, 169.1, 165.9, 72.9, 61.8, 51.1, 41.9, 24.9 23.0, 22.1, 20.7, 14.3; IR (cm⁻¹) 3308, 2961, 2873, 1758, 1656, 1541, 1203, 1058.

### EXAMPLE 8

### Preparation of 1N,4N-di[1-carbetoxy-(1S)-ethyl]-2,3-diacetoxy-(2S,3S)-butanediamide (Compound 10a)

Compound (10a) (1.57 g, 3.63 mmols) was obtained from 2,3-diacetoxy-(2S,3S)-butanedioic acid(6) (1.00 g, 4.27 mmols) and ethyl ester of L-alanine (1.21 g, 10.3 mmols), by the same procedure described for obtaining (20), with 85% yield, as a white solid. Analytically pure samples may be obtained by recrystallization in AcOEt/hexane: PF 170-171° C. [α]_{D}=−2.5 (c=0.99, CH₂Cl₂). ¹H RMN (CDCl₃, 200 MHz) δ6.97 (d, J=6.8 Hz, 1H), 5.63 (s, 1H), 4.48 (m, 1H), 4.17 (q, J=7.1 Hz, 2H), 2.14 (s, 3H), 1.37 (d, J=7.1 Hz, 3H), 1.24 (t, J=7.1 Hz, 3H); ¹³C RMN (CDCl₃, 50 MHz) δ172.6, 169.0, 165.6, 72.3, 61,9, 48.4, 20.6, 18.4, 14.2, IR (cm⁻¹) 3372, 3318, 2968, 1748, 1739, 1663, 1537, 1261, 1202, 1032.

### EXAMPLE 9

### Preparation of 1N,4N-di[1-carbetoxy-2-(1H-3-indoyl)-(1S)-ethyl]-2,3-diacetoxy-(2S,3S)-butanediamide (Compound 10 g)

Compound (10 g) (2.20 g, 3.33 mmols) was obtained from 2,3-diacetoxy-(2S,3S)-butanedioic acid(6) (1.00 g, 4.27 mmols) and ethyl ester of L-tryptophan (2.39 g, 10.3 mmols), by the same method described for obtaining (20), with 78% yield, after flash chromatography with silica gel (AcOEt/hexane-4:6), as a violet solid, PF 103-105° C. [α]_{D}=−41.7 (c=1.07, CH₂Cl₂), ¹H RMN (CDCl₃, 200 MHz) δ8.58 (s, 1H), 7.37 (d, J=7.3 Hz, 1H), 7.08 (d, J=7.2 Hz, 1H), 6.99 (m, 4H), 5.53 (s, 1H), 4.67 (m, 1H), 3.79 (q, J=7.0 Hz, 2H), 3.11 (m, 2H), 1.62 (s, 3H) 0.91 (t, J=7,0 Hz, 3H); ¹³C RMN (CDCl₃, 50 MHz) δ171.5, 169.7, 166.1, 136.2, 127.5, 123.6, 122.0, 119.4, 118.4, 111.5, 109.2, 72.2, 61.8, 53.0, 27.7, 20.2, 13.9; IR (cm⁻¹) 3406, 3058, 2981, 1740, 1673, 1525, 1205, 744.

### EXAMPLE 10

### Preparation of 1N,4N-di[-carbetoxy-2-phenyl-(1S)-ethyl]-2,3-diacetoxy-(2S,3S)-butanediamide (Compound 10e)

Compound (10e) (2.37 g, 4.06 mmols) was obtained from 2,3-diacetoxy-(2S,3S)-butanedioic acid(6) (1.00 g, 4.27 mmols) and ethyl ester of L-phenylalanine (1.99 g, 10.3 mmols), by the same procedure described for obtaining (20), with 95% yield, as a white solid. Analytically pure samples may be obtained by recrystallization in AcOEt/hexane: PF 159-160° C. [α]_{D}=+55.2 (c=1.16, CH₂Cl₂). ¹H RMN (CDCl₃, 200 MHz) δ7.45 (m, 5H), 6.98(d, J=7.5 Hz, 1H), 5.86 (s, 1H), 5.02 (m, 1H), 4.32 (q, J=7.2 Hz, 2H), 3.35 (m, 2H), 2.33 (s, 3H), 1.43 (t, J=7.2 Hz, 3H); ¹³C RMN (CDCl₃, 50 MHz) δ170.9, 169.1, 165.8, 136.8, 129.5, 128.7, 127.3, 72.2, 61.8, 53.5, 38.7, 20.6, 14.2; IR (cm⁻¹) 3353, 3333, 3086, 3033, 2983, 1755, 1663, 1536, 1211, 1066, 748, 701.

### EXAMPLE 11

### Preparation of 1N,4N-di[1-carbetoxy-2-phenyl-(1S)-ethyl]-2,3-diacetoxy-(2R,3R)-butanediamide (Compound 18)

Compound (18) (2.37 g, 4.06 mmols) was obtained from 2,3-diacetoxy-(2R,3R)-butanedioic acid(17) (1.00 g, 4.27 mmols) and ethyl ester of L-phenylalanine (1.99 g, 10.3 mmols), by the same procedure described for obtaining (20), with 95% yield, as a white solid. Analytically pure samples may be obtained by recrystallization in AcOEt/hexane: PF 141-142° C. [α]_{D}=−25.9 (c=1.20, CH₂Cl₂). ¹H RMN (CDCl₃, 200 MHz) δ7.35 (m, 5H), 6.49 (d, J=7.7 Hz, 1H), 5.69 (s, 1H), 4.82 (m, 1H), 4.19 (q, J=7.1 Hz, 2H), 3.10 (m, 2H), 2.03 (s, 3H), 1.27 (t, J=7.1 Hz, 3H); ¹³C RMN (CDCl₃, 50 MHz) δ171.9, 169.6, 166.8, 136.3, 130.2, 129.6, 127.3, 73.2, 62.6, 53.5, 38.5, 21,1, 15,0; IR (cm⁻¹) 3355, 3332, 3087, 3033, 2983, 1756, 1664, 1536, 1211, 1066, 748, 701.

### EXAMPLE 12

### Preparation of 1N,4N-di[1-carbetoxy-2-methyl-(1S)-propyl]-2,3-diacetoxy-(2S,3S)-butanediamide (Compound 10b)

Compound (10b) (1.94 g, 3.97 mmols) was obtained from 2,3-diacetoxy-(2S,3S)-butanedioic acid(6) (1.00 g, 4.27 mmols) and ethyl ester of L-valine (1.49 g, 10.3 mmols), by the same procedure method described for obtaining (20), with 93% yield, as a white solid. Analytically pure samples may be obtained by recrystallization in AcOEt/hexane: PF 147-148° C. [α]_{D}=+5.5 (c=0.99, CH₂Cl₂). ¹H RMN (CDCl₃, 200 MHz) δ6.85 (d, J=7.5 Hz, 1H), 5.56 (s, 1H), 4.44 (m, 1H) 4.14 (q, J=7.1 Hz, 2H), 2.15 (s, 3H), 2.13 (m, 1H), 1.24 (t, J=7.1 Hz, 3H), 0.89 (m, 6H); ¹³C RMN (CDCl₃, 50 MHz) δ171.4, 169.1, 166.1, 72.5, 71.0, 61.6, 57.4, 31.6, 20.7, 19.0, 17.9 14.3; IR (cm⁻¹) 3373, 3318, 2966, 1747, 1740, 1666, 1537, 1261, 1202, 1032.

### EXAMPLE 13

### Preparation of 1N,4N-di[1-carbetoxy-2-methyl-(1S,2S)-butyl]-2,3-diacetoxy-(2S,3S)-butanediamide (Compound 10d)

Compound (10d) (2.07 g, 4.01 mmols) was obtained from 2,3-diacetoxy-(2S,3S)-butanedioic acid(6) (1.00 g, 4.27 mmols) and ethyl ester of L-isoleucine (1.64 g, 10.3 mmols), by the same procedure described for obtaining (20), with 94% yield, as a white solid. Analytically pure samples may be obtained by recrystallization in AcOEt/hexane: PF 137-138° C. [α]_{D}=−8.53 (c=0.98, CH₂Cl₂). ¹H RMN (CDCl₃, 200 MHz) δ7.50 (d, J=8.0 Hz, 1H), 4.80 (d J=7.9, 1H), 4.48 (m, 1H), 4.35 (d, J=7.9, 1H), 4.19 (q, J=7.1 Hz, 2H), 2.0 (s, 3H), 1.88 (m, 1H), 1.30 (m, 5H), 0.89 (m, 6H); ¹³C RMN (CDCl₃, 50 MHz) δ173.7, 171.0, 169.8, 70.9, 61.5, 56.4, 37.9, 25.1, 15.5, 14.3, 11.7; IR (cm⁻¹) 3331, 2970, 2937, 1759, 1747, 1665, 1536, 1260, 1202, 1056.

### EXAMPLE 14

### Preparation of 1N,4N-di[2-(4-hydroxyphenyl)-1-carbetoxy-(1S)-ethyl]-2,3-diacetoxy-(2S,3S)-butanediamide (Compound 10f)

Compound (10f) (2.34 g, 3.80 mmols) was obtained from 2,3-diacetoxy-(2S,3S)-butanedioic acid(6) (1.00 g, 4.27 mmols) and ethyl ester of L-tyrosine (2.15 g, 10.3 mmols), by the same procedure described for obtaining (20), with 94% yield, after flash chromatography with silica gel (CH₂Cl₂: CH₃OH−96:4), as a crystalline white solid, PF 210-211° C., [α]_{D}=−35.84 (c=1.02, CH₃OH); ¹H RMN (DMSO-d₆, 200 MHz) δ9.24 (s, 1H), 8.35 (d, J=7.7, 1H), 6.97 (d, J=8.2 Hz, 2H), 6.64 (d, J=8.2, 2H), 5.50 (s, 1H), 4.36 (m, 1H), 4.00 (q, J=7.0 Hz, 2H), 2.85 (m, 2H), 1.94 (s, 3H), 1.09 (t, J=7.0 Hz); ¹³C RMN (DMSO-d₆, 50 MHz) δ171,4, 169.7, 166.2, 156.5, 130.4, 127.4, 115.5, 72.1, 61.2, 54.3, 36.9, 21.0, 14.5; IR (cm⁻¹) 3353, 3333, 3301, 3086, 3033, 2983, 1755, 1663, 1536, 1211, 1066, 748, 701.

### EXAMPLE 15

### Preparation of 1N,4N-dibenzyl-2,3-dihydroxy-(2R,3R)-butanediamide (Compound 21)

Sulphuric acid (0,5 ml) was added to a diamide solution (20) (0.52 g, 1.25 mmol) in absolute ethanol (50 ml) at room temperature and the mixture was kept under magnetic stirring at reflux temperature for 4 h. After cooling, the solvent was concentrated under vacuum to half the original volume, and had AcOEt (70 ml) added. The mixture was extracted with aqueous NaOH at 5% (20 ml) and aqueous 1N HCl (20 ml). The organic phase was washed with NaCl, dried with anhydrous sodium sulphate and evaporated under vacuum, providing the diol (21) (0.34 g, 10.04 mmol) at 83% yield, as a crystalline solid. PF: 198-200° C., [α]_{D}=−14.8 (c=1.20, CH₃OH), ¹H RMN (DMSO-d₆, 200 MHz) δ8.04 (m, 1H), 7.08 (m, 5H), 5.52 (d, J=7.0 Hz), 4.16 (m, 2H), 3.13 (s, 1H); ¹³C RMN (DMSO-d₆, 50 MHz) δ172.6, 139.9, 128.6, 127.6, 127.1, 73.2, 42.36; IR (cm⁻¹) 3362, 3316, 3086, 3034, 2927, 1628, 1546, 1095, 742, 697.

### EXAMPLE 16

### Preparation of 1N,4N-di[1-phenyl-(1S)-ethyl]-2,3-dihydroxy-(2S,3S)-butanediamide (Compound 1b)

Compound (1b) (0.36 g, 1.03 mmol) was obtained from the derivative (8b) (0.55 g, 1.25 mmol), by the same procedure described for obtaining (21) with 82% yield as a crystalline solid. PF: 130-131° C., [α]_{D}=−91.3 (c=1.03, CH₂Cl₂). ¹H RMN (CDCl₃, 200 MHz) δ7.29 (m, 6H), 5.20 (br s, 1H), 5.06 (dq, J=7.4, 7.0 Hz, 1H), 4.23 (s, 1H), 1.51 (d, J=7.0 Hz, 3H); ¹³C RMN (CDCl₃, 50 MHz) δ173.0, 142.3, 128.8, 127.7, 126.1, 70.3, 49.0, 21.9; IR (cm⁻¹) 3413, 3332, 3087, 3032, 2973, 1658, 1526, 1140, 1063, 763, 698.

### EXAMPLE 17

### Preparation of 1N,4N-di[1-phenyl-(1R)-ethyl]-2,3-dihydroxy-(2S,3S)-butanediamide (Compound 1c)

Compound (1c) (0.36 g, 1.03 mmol) was obtained from the derivative (8c) (0.55 g, 1.25 mmol), by the same procedure described for obtaining (21) with 82% yield as a crystalline solid. PF:144-146° C., [α]_{D}=−46.5 (c=1.12, CH₂Cl₂), ¹H RMN (CDCl₃, 200 MHz) δ7.34 (d, J=7.8 Hz, 1H), 7.21 (m, 5H), 5.32 (br s, 1H), 5.04 (m, 1H), 4.31 (s, 1H), 1.50 (d, J=7.0 Hz, 3H); ¹³C RMN (CDCl₃, 50 MHz) δ173.1, 142.3, 128.9, 127.6, 126.0, 70.5, 48.8, 22.0; IR (cm⁻¹) 3386, 3342, 3194, 2985, 2964, 1661, 1642, 1532, 1443, 1139, 1077, 763, 700.

### EXAMPLE 18

### Preparation of 1N,4N-dibenzyl-2,3-dihydroxy-(2S,3S)-butanediamide (Compound 1a)

Compound (1a) (0.34 g, 1.04 mmol) was obtained from the derivative (8a) (0.52 g, 1.25 mmol), by the same procedure described for obtaining (21) with 83% yield as a crystalline solid. PF: 198-200° C., [α]_{D}=+15.2 (c=1.20, CH₃OH), ¹H RMN (DMSO-d₆, 200 MHz) δ8.24 (m, 1H), 7.25 (m, 5H), 5.73 (d, J=7.0 Hz), 4.29 (m, 2H), 3.38 (s, 1H); ¹³C RMN (DMSO-d₆, 50 MHz) δ172.7, 139.9, 128.8, 127.9, 127.1, 73.3, 42.4; IR (cm⁻¹) 3360, 3314, 3283, 3086, 2926, 1535, 1627 1545, 1095, 742, 695.

### EXAMPLE 19

### Preparation of 1N,4N-di[1-carbetoxy-3-methyl-(1S)-butyl]-2,3-dihydroxy-(2S,3S)-butanediamide (Compound 3c)

Compound (3c) (0.44 g, 1.01 mmol) was obtained from the derivative (10c) (0.64 g, 1.25 mmol), by the same procedure described for obtaining (21) with 81% yield as a colourless oil. [α]_{D}=−21.6 (c=1.76, CH₂Cl₂), ¹H RMN (CDCl₃, 200 MHz) δ7.35 (d, J=8.0 Hz, 1H), 4.80 (br s, 1H), 4.51 (m, 1H), 4.33 (s, 1H), 4.17 (q, J=7.1 Hz, 2H), 1.62 (m, 3H), 1.25 (t, J=7.1 Hz, 3H), 0.92 (m, 6H); ¹³C RMN (CDCl₃, 50 MHz) δ173.4, 172.0, 70.7, 61.5, 50.6, 41.3, 24.8, 22.7, 21.95, 14.1; IR (cm⁻¹) 3388, 2960, 2872, 1724, 1651, 1533, 1357, 1202, 1154.

### EXAMPLE 20

### Preparation of 1N,4N-di[1-carbetoxy-(1S)-ethyl]-2,3-dihydroxy-(2S,3S)-butanediamide (Compound 3a)

Compound (3a) (0.26 g, 0.75 mmol) was obtained from the derivative (10a) (0.54 g, 1.25 mmol), by the same procedure described for obtaining (21) with 60% yield as a crystalline solid. PF: 102-103° C., [α]_{D}=−2.53 (c=0.98, CH₂Cl₂), ¹H RMN (CDCl₃, 200 MHz) δ7.58 (d, J=7.4 Hz, 1H), 4.48 (m, 1H), 4.39 (m, 1H), 4.17 (q, J=7.1 Hz, 2H), 3.92 (9br s, 1H), 1.38 (d, J=7.1 Hz, 3H), 1.24 (t, J=7,1 Hz, 3H); ¹³C RMN (CDCl₃, 50 MHz) δ173.0, 172.3, 71.1, 61.8, 48.2, 18.2, 14.2, IR (cm⁻¹) 3388, 2960, 2872, 1724, 1651, 1533, 1357, 1202, 1154.

### EXAMPLE 21

### Preparation of 1N,4N-di[1-carbetoxy-2-phenyl-(1S)-ethyl]-2,3-dihydroxy-(2S,3S)-butanediamide (Compound 3e)

Compound (3e) (0.51 g, 1.02 mmol) was obtained from the derivative (10e) (0.75 g, 1.28 mmol), by the same procedure described for obtaining (21) with 80% yield as a crystalline solid. PF: 138-140° C., [α]_{D}=+78.5 (c=1.08, CH₂Cl₂), ¹H RMN (CDCl₃, 200 MHz) δ7.41 (d, J=7.5 Hz, 1H), 7.20 (m, 5H), 4.75 (m, 1H), 4.57 (br s, 1H), 4.26 (br s, 1H) 4.07 (q, J=7.2 Hz, 2H), 3.00 (m, 2H), 1.14 (t, J=7.2 Hz, 3H); ¹³C RMN (CDCl₃, 50 MHz) δ170.8, 135.7, 129.6, 128.8, 127.4, 71.0, 61.9, 53.2, 38.3, 14.3; IR (cm⁻¹) 3448, 3384, 3347, 3062, 3030, 2979, 1729, 1658, 1625, 1531, 1209, 1138, 700, 609.

### EXAMPLE 22

### Preparation of 1N,4N-di[1-carbetoxy-2-(1H-3-indoyl)-(1S)-ethyl]-2,3-dihydroxy-(2S,3S)-butanediamide (Compound 3g)

Compound (3g) (0.42 g, 0.73 mmol) was obtained from the derivative (10g) (0.83 g, 1.25 mmol), by the same procedure described for obtaining (21) with 55% yield as a crystalline solid. PF:114-116° C., [α]_{D}=+17.9 (c=0.89, CH₃OH), ¹H RMN (CDCl₃, 200 MHz) δ10.92 (s, 1H), 7.78 (d, J=7.7 Hz, 1H), 7.50 (d, J=7.2 Hz, 1H), 6.99 (m, 4H), 5.99 (d, J=7.1 Hz, 1H), 4.63 (m, 1H), 4.33 (d, J=7.1 Hz, 1H), 3.97 (q, J=7.0 Hz, 2H), 3.21 (m, 2H), 1.07 (t, J=7.0 Hz, 3H); ¹³C RMN (CDCl₃, 50 MHz) δ172.2, 171.8, 136.5, 127.7, 124.5, 121.5, 118.9, 118.6, 111.9, 109.0, 73.0, 61.2, 52.9, 27.8, 14.3; IR (cm⁻¹) 3389, 3316, 3060, 2977, 2928, 1727, 1650, 1538, 1220, 1106, 736.

### EXAMPLE 23

### Preparation of 1N,4N-di[1-carbetoxy-2-phenyl-(1S)-ethyl]-2,3-dihydroxy-(2R,3R)-butanediamide (Compound 19)

Compound (19) (0.51 g, 1.02 mmol) was obtained from the derivative (18) (0.75 g, 1.28 mmol), by the same procedure described for obtaining (21) with 80% yield as a colourless oil. [α]_{D}=+93.1 (c=0.99, CH₂Cl₂), ¹H RMN (CDCl₃, 200 MHz) δ7.35 (d, J=7.5 Hz, 1H), 7.12 (m, 5H), 4.70 (m, 1H), 4.30 (s, 1H), 4.07 (q, J=7.2 Hz, 2H), 3.68 (br s, 1H), 3.05 (m, 2H), 1.14 (t, J=7.2 Hz, 3H); ¹³C RMN (CDCl₃, 50 MHz) δ172.4, 171.3, 135.8, 129.3, 128.8, 127.3, 71.8, 61.9, 53.5, 37.7, 14.2; IR (cm⁻¹) 3448, 3384, 3347, 3062, 3030, 2979, 1729, 1658, 1625, 1531, 1209, 1138, 700, 609.

### EXAMPLE 24

### Preparation of 1N,4N-di[1-carbetoxy-2-methyl-(1S)-propyl]-2,3-dihydroxy-(2S,3S)-butanediamide (Compound 3b)

Compound (3b) (0.41 g, 1.03 mmol) was obtained from the derivative (10b) (0.61 g, 1.25 mmol), by the same procedure described for obtaining (21) with 82% yield as a colourless oil. [α]_{D}=−2.3 (c=1.29, CH₂Cl₂), ¹H RMN (CDCl₃, 200 MHz) δ7.51 (d, J=7.5 Hz, 1H), 4.96 (br s, 1H), 4.41 (m, 2H), 4.07 (q, J=7.1 Hz, 2H), 2.13 (m, 1H), 1.24 (t, J=7.1 Hz, 3H), 0.87 (m, 6H); ¹³C RMN (CDCl₃, 50 MHz) δ173.4, 171.3, 71.5, 61.5, 57.1, 31.2, 19.0, 17.8 14.2; IR (cm⁻¹) 3404, 2968, 2938, 1737, 1662, 1530, 1206, 1150, 1025.

### EXAMPLE 25

### Preparation of 1N,4N-di[1-carbetoxy-2-methyl-(1S,2S)-butyl]-2,3-dihydroxy-(2S,35)-butanediamide (Compound 3d)

Compound (3d) (0.44 g, 1.01 mmol) was obtained from the derivative (10d) (0.64 g, 1.25 mmol), by the same procedure described for obtaining (21) with 81% yield as a colourless oil. [α]_{D}=67.3 (c=0.98, CH₂Cl₂), ¹H RMN (CDCl₃, 200 MHz) δ7.50 (d, J=8.0 Hz, 1H), 4.80 (d J=7.9, 1H), 4.48 (m, 1H), 4.35 (d, J=7.9, 1H), 4.19 (q, J=7.1 Hz, 2H), 1.88 (m, 1H), 1.30 (m, 5H), 0.89 (m, 6H); ¹³C RMN (CDCl₃, 50 MHz) δ173.7, 171.0, 70.9, 61.5, 56.4, 37.9, 25.1, 15.5, 14.3, 11.7; IR (cm⁻¹) 3404, 2968, 1736, 1660, 1530, 1203, 1148, 1024.

### EXAMPLE 26

### Preparation of 1N,4N-di[2-(4-hydroxyphenyl)-1-carbetoxy-1-(1S)-ethyl]-2,3-dihydroxy-(2S,3S)-butanediamide (Compound 3f)

Compound (3f) (0.51 g, 0.96 mmol) was obtained from the derivative (10f) (0.77 g, 1.25 mmol), by the same procedure described for obtaining (21) with 77% yield as a crystalline solid. PF: 104-105° C., [α]_{D}=−13.7 (c=0.95, CH₃OH), ¹H RMN (DMSO-d₆, 200 MHz) δ9.27 (s, 1H), 7.70 (d, J=7.7, 1H), 6.99 (d, J=8.3 Hz, 2H), 6.67 (d, J=8.3, 2H), 5.87 (d, J=7.0 Hz, 1H), 4.51 (m, 1H), 4.27 (d, J=7.0, 1H)4.04 (q, J=7,0 Hz, 2H), 2,92 (m, 2H), 1.12 (t, J=7.0 Hz); ¹³C RMN (DMSO-d₆, 50 MHz) δ172.2, 171.6, 156.7, 130.8, 126.9, 115.7, 73.0, 61.2, 53.8, 36.9, 14.5; IR (cm⁻¹) 3353, 3333, 3302, 3086, 3033, 2983, 1664, 1532, 1211, 1066, 748, 701.

### EXAMPLE 27

### Preparation of 1,4-di(benzylamine)-(2R,3R)-butane-2,3-diol (Compound 22)

A solution of compound (20) (1.20 g, 2.91 mmol) was added to a suspension of LiAlH₄ (1.30 g, 34.00 mmol) in anhydrous THF (20 ml), under argon at room temperature. The reaction mixture was kept under magnetic stirring reflux temperature for 48 h. After cooling at 0° C., water (2 ml) and aqueous 10% NaOH (3 ml) were carefully added, and the mixture was maintained under stirring at room temperature for 1 h, when it was then evaporated under vacuum. The solid residue obtained was dissolved in 1N HCl (100 ml), and extracted with AcOEt (2×20 ml). Aqueous 50% NaOH at was added to the aqueous phase, until pH 10, and the product was extracted with AcOEt (5×100 ml). This organic phase was washed with saturated NaCl (50 ml), dried with anhydrous sodium sulphate and evaporated under vacuum. The resulting residue was treated in flash column chromatography with silica gel (NH₄OH conc.: CH₃OH:CH₂Cl₂ −0,25:7,75:95) and the amino alcohol (22) was obtained (0.48 g, 1.60 mmol) at 55% yield, as a crystalline solid. PF: 77-79° C., [α]_{D}=+43.0 (c=0.98, CH₂Cl₂), ¹H RMN (CDCl₃, 200 MHz) δ7.27 (m, 5H), 3.82 (d, J=2.3 Hz, 1H), 3.76 (d, J=5.9 Hz, 1H), 3.08 (dd, J=3.4, 11.2 Hz, 1H), 3.05 (br s, 1H), 2.71 (d, J=11.2 Hz, 1H); ¹³C RMN (CDCl₃, 50 MHz) δ139.5, 128.7, 128.4, 127.4, 73.1, 54.1, 53.3; IR (cm⁻¹) 3334, 3289, 3086, 3028, 2928, 2873, 1644, 1454, 1254, 1055, 740, 701.

### EXAMPLE 28

### Preparation of 1,4-di(benzylamine)-(2S,3S)-butane-2,3-diol (Compound 2a).

Compound (2a) (0.50 g, 1.66 mmol) was obtained from the derivative (8a) (1.20 g, 2.91 mmol), by the same procedure described for obtaining (22) with 57% yield as a crystalline solid. PF: 77-79° C., [α]_{D}=−42.3 (c=1.08, CH₂Cl₂), ¹H RMN (CDCl₃, 200 MHz) δ7.27 (m, 5H), 3.93 (br s, 1H), 3.81 (d, J=2.3 Hz, 1H), 3.75 (d, J=5.9 Hz, 1H), 3.08 (dd, J=3.4, 11.2 Hz, 1H), 2.71 (d, J=11.2 Hz, 1H); ¹³C RMN (CDCl₃, 50 MHz) δ139.4, 128.7, 128.3, 127.4, 73.0, 54.0, 532; IR (cm⁻¹) 3334, 3282, 3087, 3028, 2928, 2873, 1644, 1452, 1252, 1053, 742, 701.

### EXAMPLE 29

### Preparation of 1,4-di[1-phenyl-(1S)-ethylamine]-(2S,3S)-butane-2,3-diol (Compound 2b)

Compound (2b) (0.55 g, 1.69 mmol) was obtained from the derivative (8b) (1.20 g, 2.73 mmol), by the same procedure described for obtaining (22) with 62% yield as a colourless oil. [α]_{D}=+81.3 (c=1.13, CH₂Cl₂), ¹H RMN (CDCl₃, 200 MHz) δ7.09 (m, 5H), 4.03 (br s, 1H), 3.74 (s, 1H), 4,50 (q, J=6.6 Hz, 1H), 3.08 (dd, J=3.4, 12.0 Hz, 1H), 2.71 (d, J=12.0 Hz, 1H), 1.17 (d, J=6.6 Hz, 3H); ¹³C RMN (CDCl₃, 50 MHz) δ144.6, 128.7, 127.3, 126.5, 73.1, 58.3, 51.7, 23.6, IR (cm⁻¹) 3302, 3084, 3027, 2967, 2858, 1493, 1452, 1117, 1078, 763, 701.

### EXAMPLE 30

### Preparation of 1,4-di[1-phenyl-(1R)-ethylamine]-(2S,3S)-butane-2,3-diol (Compound 2c)

Compound (2c) (0.55 g, 1.69 mmol) was obtained from the derivative (8c) (1.20 g, 2.73 mmol), by the same procedure described for obtaining (22) with 62% yield as a colourless oil. [α]_{D}=−98.0 (c=0.95, CH₂Cl₂), ¹H RMN (CDCl₃, 200 MHz) δ7.19 (m, 5H), 4.06 (br s, 1H), 3.60 (m, 2H), 2.88 (dd, J=3.0, 12.0 Hz, 1H), 2.49 (d, J=12.0 Hz, 1H), 1.31 (d, J=6.5 Hz, 3H); ¹³C RMN (CDCl₃, 50 MHz) δ144.5, 128.8, 127.4, 126.9, 73.1, 58.9, 51.9, 24.8, IR (cm⁻¹) 3310, 3084, 3027, 2966, 2854, 1493, 1452, 1118, 1079, 763, 701.

### EXAMPLE 31

### Preparation of 1N,4N-di[1-carbonylhydrazine-2-phenyl-(1S)-ethyl]-2,3-dihydroxy-(2S,3S)-butanediamide (Compound 14e)

Hydrazine hydrate (0.8 ml, 0.80 g, 16 mmol) was added to a solution of (10e)(1.17 g, 2.00 mmol) in 2 ml of DMF and 8 ml of absolute ethanol, under magnetic stirring at room temperature, and the mixture was kept under these conditions for 24 h, resulting in the formation of a precipitate. The reaction medium was transferred to 40 ml of ethyl ether and filtered in a sintered glass funnel, washed with ethyl ether (30 ml), and being collected from hydrazide (14e) (0.59 g, 1.34 mmol) at 67% yield. ¹H RMN (DMSO-d6, 200 MHz) δ11.12 (d, J=3.6 Hz, 1H), 7.72 (m, 1H), 7.45 (d, J=5.2 Hz, 1H), 7.20 (m, 5H), 5.83 (m, 1H), 5.03 (m, 0.5H), 4.54 (m, 0.5H), 4.27 (m, 1H), 4.26 (br s, 1H), 2.98 (m, 2H); ¹³C RMN (DMSO-d6, 50 MHz) δ171.3, 166.7, 137.4, 129.9, 128.7, 126.9, 73.0, 53.0, 50.8, 37.8, 18.8; IR (cm⁻¹) 3375, 3273, 3217, 3078, 2924, 1668, 1654, 1537, 1378, 763, 702.

### EXAMPLE 32

### Preparation of 1N,4N-di[1-carbonylbenzylamine-2-phenyl-(1S)-ethyl]-2,3-dihydroxy-(2S,3S)-butanediamide (Compound 15)

Hydrazyde (14e) (0.11 g, 1.26 mmol) was dissolved in a solution containing 0.88 ml of glacial acetic acid, 1.9 ml of 5N HCl and 3 ml of water at 0° C. Then sodium nitrite (0.037 g, 0.54 mmol) dissolved in a small quantity of water (circa 1 ml) was added and this mixture was kept under magnetic stirring at 0° C. for 30 minutes. The azide precipitate was extracted with iced AcOEt (20 ml), washed with iced water (10 ml), iced 5% sodium bicarbonate (10 ml) and iced water (10 ml) dried with anhydrous sodium sulphate and added to a solution of benzylamine (1,08 mmol) in 10 ml of AcOEt. The reaction medium was kept under magnetic stirring at 4° C. during 48 h. Solvent was removed under vacuum, and the residue was washed with 1N HCl (30 ml), 5% NaOH (20 ml) and water (30 ml), providing the dibenzylamide (15) (0.128 g, 0.20 mmol) at 79% yield. ¹H RMN (DMSO-d6, 200 MHz) δ8.53 (m, 1H), 7.73 (d, J=7.8 Hz, 1H), 7.10 (m, 10H), 5.78 (m, 1H), 4.63 (m, 1H), 4.21 (m, 3H), 2.99 (d, J=5.8 Hz, 2H); ¹³C RMN (DMSO-d6, 50 MHz) δ172.0, 170.6, 139.4, 137.5, 129.9, 128.7, 128.6, 127.7, 127.2 126.9, 73.1, 54.0, 42.6;IR (cm⁻¹) 3375, 3273, 3217, 3078, 2924, 1668, 1654, 1537, 1378, 763, 702.

### EXAMPLE 33

### Preparation of 1N,4N-di[1-carbonylhydrazine-benzylidene-2-phenyl-(1S)-ethyl]-2,3-dihydroxy-(2S,3S)-butanediamide (Compound 23)

Benzaldehyde (0.059 mg, 0.54 mmol) and 0.1 ml of an aqueous solution of 10% HCl were added to a solution of the hydrazide (14e) (0.11 g, 0.26 mmol) in 5 ml of 95% ethanol. The reaction mixture was kept under magnetic stirring at room temperature for 30 minutes. At the end of this period, 20 ml of water was added and extraction occurred with AcOEt (3×15 ml). The organic phase was dried with anhydrous Na₂SO₄, and the evaporation of the solvent and flash column chromatography with silica gel, employing CH₂Cl₂/MeOH 95:5 as eluent provided the dihydrazone (23) (0.131 g, 0.20 mmol) at 78% yield. ¹H RMN (DMSO-d6, 200 MHz) δ11.53 (s, 1H), 7.98 (s, 1H), 7.83 (d, J=2.6 Hz, 1H), 7.10 (m, 10H), 5.93 (m, 1H), 4.34 (m, 1H), 3.09 (m, 2H); IR (cm⁻¹) 3370, 3273, 3217, 3078, 2924, 1668, 1652, 1537, 1378, 762, 703.

### EXAMPLE 34

### Preparation of 1N,4N-di[1-carbonylhydrazine-2-hydroxy-benzylidene-2-phenyl-(1S)-ethyl]-2,3-dihydroxy-(2S,3S)-butanediamide (Compound 24)

2-hydroxy-benzaldehyde (0.066 mg, 0.54 mmol) and 0.1 ml of an aqueous solution of 10% HCl were added to a solution of the hydrazide (14e) (0.11 g, 0.26 mmol) in 5 ml of 95% Ethanol. The reaction mixture was kept under magnetic stirring at room temperature for 30 minutes. At the end of this period, 20 ml of water was added and extraction occurred with AcOEt (3×15 ml). The organic phase was dried with anhydrous Na₂SO₄, and the evaporation of the solvent and flash column chromatography with silica gel, employing CH₂Cl₂/MeOH 9:1 as eluent provided the dihydrazone (24) (0.125 g, 0.18 mmol) at 71% yield. ¹H RMN (DMSO-d6, 200 MHz) δ11.53 (s, 1H), 10.99 (s, 1H) 8.37 (s, 1H), 7.91 (d, J=2.6 Hz, 1H), 7.10 (m, 9H), 5.88 (m, 1H), 4.31 (m, 1H), 3.08 (m, 2H); IR (cm⁻¹) 3370, 3273, 3217, 3078, 2924, 1668, 1652, 1537, 1378, 762, 703.

### EXAMPLE 35

### Preparation Pharmacological Evaluation

The pharmacological evaluation of the derivatives obtained was undertaken using test plates with a PM1 strain cell culture, lymphocytic strain established in culture, expressing the receptors CD4+ and co-receptors C5 and R4 of the HIV-1 and producers of syncytium, incubated with isolated standard virus Z2Z6 purified by passage in cell culture PM-1, having a titer of 3.96×10² TCID₅₀/ml. The infection was accomplished by using plates having 96 wells, each containing 10⁴ cells/well, infected with a MOI (Multiplicity Of Infection) of 0.002. The compounds being evaluated were initially diluted in dimethylsulphoxide (DMSO) to a final concentration of 10 mM and subsequently diluted in base medium RPMI 1640 to 20 μM.

Nine wells of the cells infected initially with the isolated HIV Z6 were exposed to decreasing concentrations of the compounds at 20 μM by a factor of 2 (base log 2). The culture medium employed was the RPMI 1640, added with 10% bovine foetal serum, antibiotics streptavidine/penicilyne and L-glutamine. The most concentrated well had a final concentration of 100 μM, with the subsequent dilutions being as follows: 10 μM; 5 μM; 1.25 μM, 0.625 μM; 0.312 μM; 0.156 μM; 0.078 μM and 0.039 μM.

The last and tenth well was kept as a control of the infection, without the presence of the drug blank. Each line of ten wells was produced in triplicate, for posterior statistical analysis. INDINAVIR was used as control, in the same dilutions as the compounds being tested. Cytotoxic analysis was carried out on a fourth set of 10 (ten) wells with cells by applying the compounds of the present invention diluted as described hereinabove. The plates were kept in an oven with 5% CO₂, at a temperature of 37° C. and verified daily by optical phase microscopy for the analysis of the occurance of syncytia, which was confirmed on the 4^{th} day after infection.

The technique used for revealing the assay was colouration by 3-(4,5-dimethylthyazole-2-il)-2,5-diphenyl-tetrazole bromide to measure the cellular viability (MTT technique) (Nakashima et al; 1989), on the 6^{th} day after infection. After color revealing, the 96 well plate was read by using ELISA method, with a 490π absorption filter. The results were analysed using a Microsoft Excel matrix, with correction of the blanks, and plotting of the emission frequency graph of the assay (in percentage, using as the 100% standard the emission from the viable cells of the wells without infection) as measurement of cellular viability. The value of 50% of emission of the standard was considered as cut-off point for the IC₅₀ calculation. This value was attained, after plotting on the graph of the logarithmic regression curve equation, the points obtained from the IC curve prior to the formation of the plateau of the curve. The results obtained are described in Table 5.

Table 5: Pharmacological evaluation of some of the derivatives obtained.

| Compound | IC₅₀ |
|---|---|
| Indinavir | 0, 2 μM |
| (standard) | |
| 20 | >100 μM |
| 8a | >100 μM |
| 21 | >100 μM |
| 1a | >100 μM |
| 22 | >100 μM |
| 2a | >100 μM |
| 8b | >100 μM |
| 8c | >100 μM |
| 1b | >100 μM |
| 1c | >100 μM |
| 11b | >100 μM |
| 11c | >100 μM |
| 2b | 2 μM |
| 2c | 4 μM |
| 18 | >100 μM |
| 19 | >100 μM |
| 10e | 50 μM |
| 3e | >100 μM |
| 3b | >100 μM |
| 3c | 10 μM |
| 3d | >100 μM |
| 3g | >100 μM |
| 3f | 50 μM |
| 3a | >100 μM |
| 14e | >100 μM |
| 15 | >100 μM |

Whilst the present invention has been described in terms of its preferred embodiments, it is obvious to one versed in the state of the art that various alterations and modifications are possible without diverging with the scope of the present invention, which is determined in the claims enclosed.

1. Compound characterised by possessing the following formula: 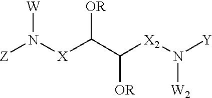
where:
Z and Y are independently selected from CHR₂R₃; CHR₄COOR₅; CHR₄CONHR₆ and CHR₄C(O)NHN═CR₇R₈
R₆ is selected from (NH₂), CHR₄COOR₅, hydrogen, aryl, substituted aryl, arylalkyl, substituted arylalkyl, heterocycles, alkyl heterocycles and lower alkyl
R₂, R₃, R₄, R₇, R₈ are independently selected from hydrogen, aryl, substituted aryl, arylalkyl, substituted arylalkyl, heterocycles, alkyl heterocycles and lower alkyl
R₅ is a lower alkyl or hydrogen
W and W₂ are independently selected from hydrogen, lower alkyl, carbonylalkyl, carbonylaryl, alkylsulphone, arylsulphone, substituted arylsulphone
R is hydrogen or a protecting group and
X and X₂ are independently selected from CH₂ and CO,
or a pro drug or a pharmaceutically acceptable salt of said compound.

2. Compound according to claim 1 characterised by Z and Y are independently (CHR₄) (COOR₅); R being hydrogen or acyl, acetyl, phosphoryl pivaloyl, t-butylacetyl, benzoyl, substituted methyl ethers, substituted ethyl ethers, or esters prepared by reacting of the hydroxyl group with a carboxylic acid group, such as, acetate, propionate or benzoate; X and X₂ being independently selected from CH₂ and CO; W and W₂ being independently selected from hydrogen, methyl, ethyl, n-propyl, iso-propyl, n-butyl, iso-butyl, sec-butyl, n-pentyl, methylsulphone, n-propylsulphone, isopropylsulphone, n-butylsulphone, isobutylsulphone, benzenesulphone, 4-methyl-benzenesulphone, 4-amino-benzenesulphone, 4-hydroxy-benzenesulphone, benzenecarbonyl, 4-methyl-benzenecarbonyl, 4-amino-benzenecarbonyl, 4-hydroxy-benzenecarbonyl, acetyl, propionyl, n-butyryl, isobutyryl, n-valeroyl or isovaleroyl.

3. Compound according to claim 2 characterised by R₅ being a lower alkyl or hydrogen; R₄ being hydrogen, aryl, substituted aryl, arylalkyl, substituted arylalkyl, heterocycle, alkyl heterocycle or lower alkyl; R being hydrogen; X and X₂ being oxygen and W and W₂ being hydrogen.

4. Compound according to claim 3 characterised by R₄ and R₅ are a lower alkyl.

5. Compound according to claim 4 characterised by R₄ is propyl and R₅ being ethyl.

6. Compound according to claim 3 characterized by R₅ is hydrogen; R₄ being hydrogen, aryl, substituted aryl, arylalkyl, substituted arylalkyl, heterocycle, alkyl heterocycle and lower alkyl; R being hydrogen; X and X₂ being oxygen and W and W₂ being hydrogen.

7. Compound according to claim 6 characterised by R₄ is a lower alkyl.

8. Compound according to claim 7 characterised by R₄ is propyl.

9. Compound according to claim 1 characterised by Z and Y are CHR₂R₃; R being hydrogen, acyl, acetyl, phosphoryl pivaloyl, t-butylacetyl, benzoyl, substituted methyl ethers, substituted ethyl ethers, or esters prepared by reacting hydroxyl group with a carboxylic acid group, such as, acetate, propionate or benzoate; X and X2 being independently selected from oxygen or hydrogen; W and W2 being independently selected from hydrogen, methyl, ethyl, n-propyl, iso-propyl, n-butyl, iso-butyl, sec-butyl, n-pentyl, methylsulphone, n-propylsulphone, isopropylsulphone, n-butylsulphone, isobutylsulphone, benzenesulphone, 4-methyl-benzenesulphone, 4-amino-benzenesulphone, 4-hydroxy-benzenesulphone, benzenecarbonyl, 4-methyl-benzenecarbonyl, 4-amino-benzenecarbonyl, 4-hydroxy-benzenecarbonyl, acetyl, propionyl, n-butyryl, isobutyryl, n-valeroyl or isovaleroyl.

10. Compound according to claim 9 characterised by R₂ and R₃ are independently selected from hydrogen, aryl, substituted aryl, arylalkyl, substituted arylalkyl, heterocycle, alkyl heterocycle or lower alkyl; R₁ being hydrogen; X being oxygen and W being hydrogen.

11. Compound according to claim 10 characterised by R₂ is an aryl and R₃ a lower alkyl.

12. Compound according to claim 11 characterised by R₂ is phenyl and R₃ is methyl.

13. Compound according to claim 9 characterised by R₂ and R₃ are independently selected from hydrogen, aryl, substituted aryl, arylalkyl, substituted arylalkyl, heterocycle, alkyl heterocycle or lower alkyl; R, X, X₂, W and W₂ being hydrogen.

14. Compound according to claim 13 characterised by R₂ is an aryl and R₃ a lower alkyl.

15. Compound according to claim 14 characterised by R₂ is phenyl and R₃ is methyl.

16. Compound according to claim 1 characterised by Z and Y are CHR₄CONHR₆; R₄ being independently selected from hydrogen, aryl, substituted aryl, arylalkyl, substituted arylalkyl, heterocycle, alkyl heterocycle and lower alkyl; R being hydrogen, acyl, acetyl, phosphoryl pivaloyl, t-butylacetyl, benzoyl, substituted methyl ethers, substituted ethyl ethers, or esters prepared by reacting hydroxyl group with a carboxylic acid group, such as acetate, propionate or benzoate; X and X₂ being independently selected from oxygen and hydrogen; W and W₂ being independently selected from hydrogen, methyl, ethyl, n-propyl, iso-propyl, n-butyl, iso-butyl, sec-butyl, n-pentyl, methylsulphone, n-propylsulphone, isopropylsulphone, n-butylsulphone, isobutylsulphone, benzenesulphone, 4-methyl-benzenesulphone, 4-amino-benzenesulphone, 4-hydroxy-benzenesulphone, benzenecarbonyl, 4-methyl -benzenecarbonyl, 4-amino-benzenecarbonyl, 4-hydroxy-benzenecarbonyl, acetyl, propionyl, n-butyryl, isobutyryl, n-valeroyl or isovaleroyl.

17. Compound according to claim 16 characterised by R₆ is (NH₂), CHR₄COOR₅, hydrogen, aryl, substituted aryl, arylalkyl, substituted arylalkyl, heterocycle, alkyl heterocycle or lower alkyl.

18. Compound according to claim 17 characterised by R₆ is (NH₂) and R₄ is arylalkyl.

19. Compound according to claim 18 characterised R₄ is benzyl.

20. Compound according to claim 17 characterised R₆ is CHR₄COOR₅.

21. Compound according to claim 20 characterised by R₅ is a lower alkyl or hydrogen, R₄ being independently selected from hydrogen, aryl, substituted aryl, arylalkyl, substituted arylalkyl, heterocycle, alkyl heterocycle or lower alkyl.

22. Compound according to claim 17 characterised by R₆ is selected from hydrogen, aryl, substituted aryl, arylalkyl, substituted arylalkyl, heterocycle, alkyl heterocycle or lower alkyl.

23. Compound according to claim 1 characterised by Z and Y are CHR₄C(O)NHN═CR₇R₈, R₄ being independently selected from hydrogen, aryl, substituted aryl, arylalkyl, substituted arylalkyl, heterocycle, alkyl heterocycle or lower alkyl; R is hydrogen, acetyl, phosphoryl pivaloyl, t-butylacetyl, benzoyl, substituted methyl ethers, substituted ethyl ethers, or esters prepared by reacting hydroxyl group with a carboxylic acid group, such as, acetate, propionate or benzoate; X and X₂ being independently selected from oxygen and hydrogen, W and W₂ being independently selected from hydrogen, methyl, ethyl, n-propyl, iso-propyl, n-butyl, iso-butyl, sec-butyl, n-pentyl, methylsulphone, n-propylsulphone, isopropylsulphone, n-butylsulphone, isobutylsulphone, benzenesulphone, 4-methyl-benzenesulphone, 4-amino-benzenesulphone, 4-hydroxy-benzenesulphone, benzenecarbonyl, 4-methyl-benzenecarbonyl, 4-amino-benzenecarbonyl, 4-hydroxy-benzenecarbonyl, acetyl, propionyl, n-butyryl, isobutyryl, n-valeroyl and isovaleroyl.

24. Compound according to claim 23 characterised by R₇ and R₈ are independently selected from hydrogen, aryl, substituted aryl, arylalkyl, substituted arylalkyl, heterocycle, alkyl heterocycle or lower alkyl.

25. Compound according to claim 1 characterised of being selected from the group consisting of:
1N,4N-dibenzyl-2,3-diacetoxy-(2R,3R)-butanediamide;
1N,4N-dibenzyl-2,3-diacetoxy-(2S,3S)-butanediamide;
1N,4N-di[1-phenyl-(1S)-ethyl]-2,3-diacetoxy-(2S,3S)-butanediamide;
1N,4N-di[1-phenyl-(1R)-ethyl]-2,3-diacetoxy-(2S,3S)-butanediamide;
1N,4N-di[1-carbetoxy-3-methyl-(1S)-butyl]-2,3-diacetoxy-(2S,3S)-butanediamide;
1N,4N-di[1-carbetoxy-(1S)-ethyl]-2,3-diacetoxy-(2S,3S)-butanediamide;
1N,4N-di[1-carbetoxy-2-(1H-3-indoyl)-(1S)-ethyl]-2,3-diacetoxy-(2S,3S)-butanediamide;
1N,4N-di[1-carbetoxy-2-phenyl-(1S)-ethyl]-2,3-diacetoxy-(2S,3S)-butanediamide;
1N,4N-di[1-carbetoxy-2-phenyl-(1S)-ethyl]-2,3-diacetoxy-(2R,3R)-butanediamide;
1N,4N-di[1-carbetoxy-2-methyl-(1S)-propyl]-2,3-diacetoxy-(2S,3S)-butanediamide;
1N,4N-di[1-carbetoxy-2-methyl-(1S,2S)-butyl]-2,3-diacetoxy-(2S,3S)-butanediamide;
1N,4N-di[2-(4-hydroxyphenyl)-1-carbetoxy-(1S)-ethyl]-2,3-diacetoxy-(2S,3S)-butanediamide;
1N,4N-dibenzyl-2,3-dihydroxy-(2R,3R)-butanediamide;
1N,4N-di[1-phenyl-(1S)-ethyl]-2,3-dihydroxy-(2S,3S)-butanediamide;
1N,4N-di[1-phenyl-(1R)-ethyl]-2,3-dihydroxy-(2S,3S)-butanediamide;
1N,4N-dibenzyl-2,3-dihydroxy-(2S,3S)-butanediamide;
1N,4N-di[1-carbetoxy-3-methyl-(1S)-butyl]-2,3-dihydroxy-(2S,3S)-butanediamide;
1N,4N-di[1-carbetoxy-(1S)-ethyl]-2,3-dihydroxy-(2S,3S)-butanediamide;
1N,4N-di[1-carbetoxy-2-phenyl-(1S)-ethyl]-2,3-dihydroxy-(2S,3S)-butanediamide;
1N,4N-di[1-carbetoxy-2-(1H-3-indoyl)-(1S)-ethyl]-2,3-dihydroxy-(2S,3S)-butanediamide;
1N,4N-di[1-carbetoxy-2-phenyl-(1S)-ethyl]-2,3-dihydroxy-(2R,3R)-butanediamide;
1N,4N-di[1-carbetoxy-2-methyl-(1S)-propyl]-2,3-dihydroxy-(2S,3S)-butanediamide;
1N,4N-di[1-carbetoxy-2-methyl-(1S,2S)-butyl]-2,3-dihydroxy-(2S,3S)-butanediamide;
1N,4N-di[2-(4-hydroxyphenyl)-1-carbetoxy-(1S)-ethyl]-2,3-dihydroxy-(2S,3S)-butanediamide;
1,4-di(benzylamine)-(2R,3R)-butane-2,3-diol;
1,4-di(benzylamine)-(2S,3S)-butane-2,3-diol;
1,4-di[1-phenyl-(1S)-ethylamine]-(2S,3S)-butane-2,3-diol;
1,4-di[1-phenyl-(1R)-ethylamine]-(2S,3S)-butane-2,3-diol;
1N,4N-di[1-carbonylhydrazine-2-phenyl-(1S)-ethyl]-2,3-dihydroxy-(2S,3S)-butanediamide;
1N,4N-di[1-carbonylbenzylamine-2-phenyl-(1S)-ethyl]-2,3-dihydroxy-(2S,3S)-butanediamide;
1N,4N-di[1-carbonylhydrazine-benzylidene-2-phenyl-(1S)-ethyl]-2,3-dihydroxy-(2S,3S)-butanediamide;
1N,4N-di[1-carbonylhydrazine-2-hydroxy-benzylidene-2-phenyl-(1S)-ethyl]-2,3-dihydroxy-(2S,3S)-butanediamide;
or a pro drug or a pharmaceutically acceptable salt of said compound.

26. Pharmaceutical composition characterised by including as active ingredient an efficient quantity of one of the compounds in accordance with claim 1 or 25 and a pharmaceutically acceptable vehicle.

27. Pharmaceutical composition according to claim 26 characterised by the active ingredient is present at a concentration varying between 0.1 and 99% of the weight of the formulation.

28. Pharmaceutical composition according to claim 27 characterised by the active ingredient is present at a concentration varying between 0.25 and 99% of the weight of the formulation.

29. Use of one of the compounds of claim 1 or 25 in the preparation of a drug adequate for the treatment of infections caused by HIV.

30. Use of one of the compounds of claim 1 or 25 in the preparation of a drug adequate for the use in inhibiting the HIV protease.
